(19) United States
(12) Patent Application Publication
(10) Pub. No.: US 2004/0058380 A1
(43) Pub. Date: Mar. 25, 2004

(54) Surface imprinting: integration of molecular recognition and transduction

(76) Inventors: Kalle Levon, New York, NY (US); Yanxiu Zhou, Brooklyn, NY (US); Bin Yu, Brooklyn, NY (US)

Correspondence Address: STRAUB & POKOTYLO, 620 TINTON AVENUE, BLDG. B, 2ND FLOOR, TINTON FALLS, NJ, 07724, US

(21) Appl. No.: 10/242,590

(22) Filed: Sep. 12, 2002

(51) Int. Cl. G01N 33/53; C12P 19/46; C12M 1/34

(52) U.S. Cl. 435/7.1; 435/287.2; 205/777.5

(57) ABSTRACT

Surface-molecularly imprinted sensors were fabricated for detecting ionic molecules. Target molecules are recognized by the combination of a hydrophobic interaction with the imprinted polymer layer to provide specificity and an electrostatic interaction to provide sensitivity Coupling surface imprinting techniques with an electrochemical detection method, such as potentiometry, allows specific recognition of target molecules and translation of the recognition event into an output signal by the sensor.

### § 0. FEDERALLY SPONSORED RESEARCH

This invention was made with Government support and the Government has certain rights in the invention as provided for by contract number 0660076225 awarded by DARPA.

### § 1. BACKGROUND

§ 1.1 Field of the Invention

The present invention concerns new concepts in sensing, particularly integrating molecular recognition processes and sensor transduction, and their application to detecting ionic activity.

§ 1.2 Related Art

A chemical sensor is a device that transforms chemical information, such as the concentration of a specific sample component or total composition analysis, into an analytically useful signal. Chemical sensors typically contain two basic components connected in series: a chemical (molecular) recognition system (receptor) and a physico-chemical transducer (See, e. g., D. R. Thevenot, Biosensors & Bioelectronics 16, 1210-131 (2001). Janata and Bezegh define a chemical sensor as “a device which furnishes the user with information about its environment; it consists of a physical transducer and a chemically selective layer” (J. Janata, A. Bezegh, Anal. Chem. 60, 62R (1988)).

Molecular recognition refers to the selective binding of a probe molecule to a molecular receptor. This binding interaction relies on both non-covalent intermolecular chemical interactions and steric compatibility, such as size or shape inclusion (Buckingham. A. D. In Principles of Molecular Recognition. Buckinghamk, A. D., Legon A. C., Roberts, S. M. Eds.; Blkackie Academic & Professional, London, 1993; pp 1-16.). Therefore, both chemical and physical (e.g., size or shape inclusion) recognition should be considered during a molecular recognition process. Most efforts in sensor development, however, have conformed to Janata and Bezegh's definition of the chemical sensor and as such sought only chemical recognition mechanisms.

Molecular imprinting, a surface imprinting method, is a technique that allows cavities or imprints to be made in a polymer. Growing interest in molecular imprinting may be attributed to its recent success in collecting properties for both enthalpic and entropic contributions of the binding process (Molecularly imprinted polymers, edited by B. Sellergren, Elsevier, 2001). However, this technique may still limited in terms of chemical sensing by the fact that its transduction mechanism is separated from the binding event. The natural process, in contrast, integrates recognition and transduction, as in the function of an ion channel in a natural membrane, for example. Thus, mimicking nature by combining the recognition and transduction processes by imprinting the template on the surface of the transducer is a target of intensified research.

Mesoporous tin dioxide, SnO₂, has been used to fabricate solid state semiconductor sensors for reducing gases (G. -J. Li, S,. Kawi, Talanta 1998, 45, 759-766). In such a sensor, the reducing gases in contact with SnO₂ react with oxygen species such as O⁻ or O₂⁻ to remove them from the SnO₂ surfaces. This change in the surface structure causes an increase in the conductivity, making these types of semiconductor surfaces attractive for building the possible surface imprinting methods.

Forming monolayers of bonded organosilane reagents under anhydrous reaction conditions was discovered by Untereker et. al. with the modification of SnO₂, TiO₂ and glassy carbon electrodes (Untereker, D. F., Lennox, J. C., Wier, L. M., Moses, P. R., Murray, R. W., J. Electroanal. Chem. 1977, 81, 309-318). Sagiv et. al. were able to form similar oleophobic monomolecular films containing mixed monolayers of more than one component on similar polar surfaces from anhydrous organic solutions (Sagiv, J. JACS 1980, 102, 92-98). Covalent binding between the monolayer surface and intralayer cross-linking was shown to result in an unusual mechanical, chemical and electrical stability (L. Netzer, J. Sagiv, JACS 1983, 105, 674-676). The covalently bonded silane monolayers were shown to be perfectly stable under conditions which cause a major deterioration of the structure of fatty acid films (J. Gun, R. Iscovici, J. Sagiv, Journal Colloid and Interface Science, 1984, 101, 201-213). Sagiv et. al. also studied the thermal behavior of the monolayers prepared by covalent, ionic and physical bonds and found that with the exception of the covalently bound OTS (S. R. Cohen, R. Naaman, J. Sagiv, J. Phys. Chem. 1986, 90, 3045-3056), all other monolayers underwent a large melting transition around 110° C. The heating caused only slight disorientation of the chains (by FTIR investigation) in octadecyltrichlorosilane (OTS) and no sharp melting point was observed. The significant structural reversibility upon recooling was attributed to the stable OTS monolayers as the immobilization of the head groups by covalent intralayer can be expected to prevent melting of such films.

The fact that the mixed monolayers contained both physisorbed and chemisorbed components made removing the physisorbed components possible. Sagiv proposed that the resulting skeleton monolayers have holes of molecular dimensions which may be used for free adsorption sites. To investigate his proposal, mixed monolayers were formed in the presence of surfactant dyes which were only adsorbed on the surface in contrast to the monolayers which were covalently linked. The dye molecules were washed away, leaving holes in the polymerized silane network (Sagiv, J., Israel Journal of Chemistry, 1979, 18, 346-353). Sagiv's work can be considered the first surface molecular imprinting experiment.

Tabushi et al have applied Sagiv's molecular imprinting concept to imprinting alkanes in the above mentioned mixed monolayers (Tabushi, I., Kurihara, K., Naka, K., Yamamura, K., Hatakeyama, H., Tetrahedron Letters, 1987, 28 (37), 4299-4302 and Yamamura, K., Hatakeyama, H., Naka, K., Tabushi, I., Kurihara, K., J. Chem. Soc., Chem. Commun. 1988, 79-81). In their studies, Tabushi et al used n-hexadecane as the template molecule and co-implanted the template with the ODS molecules on SnO₂ surface. The formed monolayer was able to sense molecules with alkyl tails, indicating the presence of spaces left by the template molecules. This application confirmed the recognition components but did not take advantage of the sensor's active surface properties. Detection of affinity binding by guest recognition was exemplified by Mosbach et. al. using ellipsometry for the binding measurements (Andersen, L. I., Mandenius, C. E., Mosbach, K. Tetrahedron Letters, 1988, 29 (42), 5437-5440).

Given the two processes (chemical and physical) that facilitate molecular recognition combined with advances in molecular imprinting techniques, a novel improvement in the field of sensor development would include a chemical sensor containing two basic components connected in series: a molecular recognition system (including physical and chemical recognition) and a physico-chemical transducer. Sensor designs should not consider chemical recognition between the receptor and analyte alone, but also incorporate physical recognition processes. A chemical sensor, thus, could include a molecular recognition process as well as a physico-transducer. Such sensors may be created using molecular imprinting techniques. The present invention overcomes the limitations of previous chemical sensor methodologies by applying dual (chemical and physical) recognition processes coupled with new or modified detection methods to fabricate highly selective chemical sensors for ionic molecules.

### § 2. SUMMARY OF THE INVENTION

The present invention uses surface imprinting techniques to fabricate sensors that may selectively recognize target molecules of complementary sizes, geometries, and functionalities by supporting electrolyte-, and buffer-free potentiometry for amino acids and by an alternative potentiometry for other kinds of ionic molecules.

The sensor's transducer plays an important role in the recognition process. Nano-scale sized indium tin oxide (ITO) provides a high signal to background ratio and gives higher sensitivity to the analytes than normal transducers.

The present invention investigates using ionic molecules in the preparation of modified monolayers. The advantage of such an approach is the chemical interaction with the ionic species by the semiconductor surface, which results in simultaneous signaling when molecules are recognized. The present invention involves fabricating a chemical sensor and its use for the potentiometric measurement of three exemplary template molecules: chiral amino acid derivative (N-CBZ-L-Asp), a degradation product of the nerve gas Sarin (Methylphosphonic acid, MPA), and a major component of bacterial endospore (Dipicolinic acid (pyridine-2, 6-dicarboxylic acid), DPA). Surface molecularly-imprinted sensors were used to detect dipicolinic acid and methylphosphonic acid by alternative potentiometry whereas chiral amino acid derivatives were detected by support electrolyte- and buffer-free potentiometry.

In the present invention, an OTS monolayer was covalently bound onto the ITO surface in the presence of each of the template molecules. Chiral N-CBZ-Asp compound was selected for its potential ability to be enantioselective for N-CBZ-Asp. MPA detection could be used to prove the use of nerve agents (Hirsjärvi, P.; Miettinen, J. K.; Paasivirta, J. (Editors), Identification of Degradation Products of Potential Organophosphorus Warfare Agents. An Approach for the Standardization of Techniques and Reference Data, Ministry of Foreign Affairs of Finland, Helsinki, 1980, pp 3-10, 18-30 and appendices). DPA, a major constituent of bacterial endospores (including Bacillus anthracis spores) comprising 5 to 14% of their dry weight, offers the potential for detecting biological agents (Murrell, W. G.; Warth, A. D. Spores III. American Society for Microbiology; L. L. Campbell, L. L.; Halvorson, H. O. eds.; Ann Arbor: Mich. 1965; p. 1-24. Warth, A. D. Adv. Microb. Physiol. 1978,17, 1-45).

### § 3. BRIEF DESCRIPTION OF DRAWINGS

FIG. 1 is a scheme depicting sensor fabrication using surface molecular imprinting technology.

FIG. 2 is a scheme depicting the experimental assembly of a surface-molecularly imprinted sensor used with a potentiometer.

FIG. 3 is a scheme depicting the recognition mechanism of a surface-molecularly imprinted sensor to its target molecule.

FIG. 4 is scheme depicting the equilibria of aspartic acids as a function of solution pH.

FIG. 5 is a graph that shows the potentiometric responses of N-CBZ-L-Asp on OTS/ITO electrodes (fabricated with (●) 0.8 mM OTS and 0.037 M N-CBZ-L-Asp for 3 minutes adsorption time) and without (▪) surface-molecularly imprinted cavities for N-CBZ-L-Asp.

FIGS. 6a and 6b are graphs that show the potentiometric responses of (a) an ITO glass plate and (b) a surface-molecularly imprinted sensor for N-CBZ-L-Asp for HCl at pH 3.5˜5 (◯) and at pH 2.5˜3.5 (Δ).

FIG. 7 is a graph that shows calibration curves for the potentiometric responses of the L- (●, ◯) and D-isomers (▪, □) of N-CBZ-L-Asp dissolved in water (pH 1.5-5.08) (◯, □) or dissolved in 0.1 M phosphate buffer +0.1 M KCl (pH 6.8) (●, ▪) on the N-CBZ-L-Asp surface-molecularly imprinted sensor.

FIG. 8 is a graph of the potential responses of a surface-molecularly imprinted sensor for N-CBZ-L-Asp sensor for the L- (●) and D-isomers (▪) of N-CBZ-Asp as a function of pH.

FIGS. 9a and 9b are graphs that show the enantioselective potential responses of N-CBZ-L-Asp (●) and N-CBZ-D-Asp (♦) by N-CBZ-L-Asp and N-CBZ-D-Asp sensors, respectively.

FIG. 10 is a graph that shows enantioselective potential responses for mixtures of enantiomers. (◯) N-CBZ-D-Asp detected by N-CBZ-D-Asp sensor; (+) N-CBZ-L-Asp detected by N-CBZ-D-Asp sensor; (⋄) added N-CBZ-D-Asp from initial condition of 0.0001 M N-CBZ-L-Asp and detected by N-CBZ-D-Asp sensor, (x) added N-CBZ-D-Asp from initial condition of 0.001 M N-CBZ-L-Asp and detected by N-CBZ-D-Asp sensor; (▴) added N-CBZ-L-Asp from initial condition of 0.001 M N-CBZ-D-Asp and detected by N-CBZ-D-Asp sensor.

FIG. 11 is a graph of the potential responses of N-CBZ-L-Asp (◯), N-CBZ-L-Glu (▾), L-Glu [symbol] , L-Asp (●), and L-Phe (∇) by a surface-molecularly imprinted sensor for N-CBZ-L-Asp.

FIG. 12 is a graph that shows the potentiometric responses of DPA on OTS/ITO electrodes with (●) and without (♦) surface-molecularly imprinted cavities for DPA. (Conditions: PBS buffer (pH 7.2) at 0° C. Adsorption time: 3 min. [OTS]=0.8 mM. [DPA]=0.033 M.) FIG. 13 is a graph that shows the effect of buffer pH on the response of a surface-molecularly imprinted sensor for DPA to 0.0103 M DPA in PBS buffer at 0° C.

FIG. 14 is a graph that shows the potentiometric response of a surface-molecularly imprinted sensor for DPA as a function of pH.

FIG. 15 is a graph that shows the calibration curve for a surface-molecularly imprinted sensor's potentiometric response to DPA.

FIG. 16 is a graph that shows the selectivity of a surface-molecularly imprinted sensor for DPA to its target molecule over benzoic acid.

FIGS. 17a and 17b are graphs that show the selectivity of a surface-molecularly imprinted sensor for DPA to its target molecule over inorganic compounds NaCl (a) and KCl (b).

FIGS. 18a and 18b are graphs that show the selectivity of a surface-molecularly imprinted sensor for DPA to its target molecule over inorganic compounds K₂HPO₄ (a) and NaNO₃ (b).

FIGS. 19a and 19b are graphs that show the selectivity of a surface-molecularly imprinted sensor for DPA to its target molecule over inorganic compounds NH₄NO₃ (a) and (NH₄)₂SO₄ (b).

FIGS. 20a and 20b are graphs that show the selectivity of a surface-molecularly imprinted sensor for DPA to its target molecule over inorganic compound CaCO₃ (a) and organic compound nutrient broth and tryptone (b).

FIGS. 21a and 21b are graphs that show the selectivity of a surface-molecularly imprinted sensor for DPA to its target molecule over organic compounds D-phenylalanine (a) and L-glucose (b).

FIGS. 22a and 22b are graphs that show the selectivity of a surface-molecularly imprinted sensor for DPA to its target molecule over organic compounds D(+)-malic acid (a) and sodium benzoate (b).

FIGS. 23a and 23b are graphs that show the selectivity of a surface-molecularly imprinted sensor for DPA to its target molecule over organic compounds L-tyrosine (a) and DL-tryptophan (b).

FIGS. 24a and 24b are graphs that show the selectivity of a surface-molecularly imprinted sensor for DPA to its target molecule over organic compounds riboflavin (a) and P-NAD (b).

FIGS. 25a and 25b are graphs that show the selectivity of a surface-molecularly imprinted sensor for DPA to its target molecule over biological compounds lyophilized Bacillus subtilis (a) and lyophilized Azotobacter vinelandii (b).

FIGS. 26a and 26b are graphs that show the selectivity of a surface-molecularly imprinted sensor for DPA to its target molecule over biological compounds defatted German cockroach (a) and nondefatted Eastern cottonwood pollen (b).

FIGS. 27a and 27b are graphs that show the selectivity of a surface-molecularly imprinted sensor for DPA to its target molecule over biological compounds nondefatted cultivated wheat pollen (a) and nondefatted desert ragweed pollen (b).

FIGS. 28a and 28b are graphs that show the selectivity of a surface-molecularly imprinted sensor for DPA to its target molecule over biological compounds defatted Aspergillus flavus mold (a) and defatted Cladosporium herbarum mold (b).

FIGS. 29a and 29b are graphs that show the selectivity of a surface-molecularly imprinted sensor for DPA to its target molecule over biological compounds lyophilized Aerobacter aerogenes Type I (a) and Micrococcus luteus in enriched nutrient broth (b).

FIGS. 30a and 30b are graphs that show the selectivity of a surface-molecularly imprinted sensor for DPA to its target molecule over biological compounds lyophilized Pseudomonas fluorescens Type IV (a) and Bacillus subtilis in nutrient broth (b).

FIG. 31 is a graph that shows the selectivity of a surface-molecularly imprinted sensor for DPA to its target molecule over biological compound yeast candida utiliz.

FIG. 32 is a graph that shows the simulated (line) and actual [symbol] potentiometric responses of a surface-molecularly imprinted sensor for DPA for its target molecule.

FIG. 33 is a graph that shows the potentiometric responses of MPA on OTS/ITO electrodes with (^{●}) and without [symbol] surface-molecularly imprinted cavities for DPA.

FIG. 34 is a graph that shows the effect of pH on the response of a surface-molecularly imprinted sensor for MPA to 0.2529 M MPA.

FIG. 35 is a graph that shows the potential response of a surface-molecularly imprinted sensor for MPA as a function of pH.

FIG. 36 is a graph that shows the potentiometric response of MPA sensor to ethylphosphonic acid (EPA), propylphosphonic acid (PPA) and tert-butylphosphonic acid (BPA).

FIG. 37 is a graph that compares the potentiometric response of a surface-molecularly imprinted sensor for MPA to MPA and H₃PO₄.

FIG. 38 is a graph that compares the potentiometric response of a surface-molecularly imprinted sensor for MPA to MPA and Na₃PO₄.

FIG. 39 is a graph that compares the potentiometric response of a surface-molecularly imprinted sensor for MPA to MPA and dimethoate.

FIG. 40 is a graph that compares the potentiometric response of a surface-molecularly imprinted sensor for MPA to MPA and methyl parathion.

FIG. 41 is a graph that compares the potentiometric response of a surface-molecularly imprinted sensor for MPA to MPA and phosdrin.

FIG. 42 is a graph that compares the potentiometric response of a surface-molecularly imprinted sensor for MPA to MPA and dichlorophos.

FIG. 43 is a graph that compares the potentiometric response of a surface-molecularly imprinted sensor for MPA to MPA and dibutyl chlorendate.

FIG. 44 is a graph that compares the potentiometric response of a surface-molecularly imprinted sensor for MPA to MPA and malathion.

FIG. 45 is a graph that compares the potentiometric response of a surface-molecularly imprinted sensor for MPA to MPA and thionazin.

FIG. 46 is a graph that shows the simulated (line) and actual (⋄) potentiometric responses of a surface-molecularly imprinted sensor for MPA for its target molecule.

### § 4. DETAILED DESCRIPTION

The following description is presented to enable one skilled in the art to make and use the invention, and is provided in the context of particular embodiments and methods. Various modifications to the disclosed embodiments and methods will be apparent to those skilled in the art, and the general principles set forth below may be applied to other embodiments, methods, and applications. Thus, the present invention is not intended to be limited to the embodiments and methods shown and the inventors regard their invention as the following disclosed methods, apparatus, and materials and any other patentable subject matter to the extent that they are patentable.

Sensor fabrication is described in § 4.1 below. Then, sensors operation is described in § 4.2. Thereafter, various exemplary sensors are described in § 4.3.

§ 4.1 Sensor Fabrication

Fabricating surface-imprinted sensors involves (i) co-adsorbing polymer and template molecules on the sensor's surface and (ii) removing the template molecules from the sensor's surface. Sensor fabrication in accordance with the present invention is described in § 4.1.1. Thereafter, specific methods for fabricating exemplary surface-molecularly imprinted sensors for N-CBZ-Asp, dipicolinic acid (DPA), and (methylphosphonic acid) MPA are described in § 4.1.2.

§ 4.1.1 General Sensor Fabrication

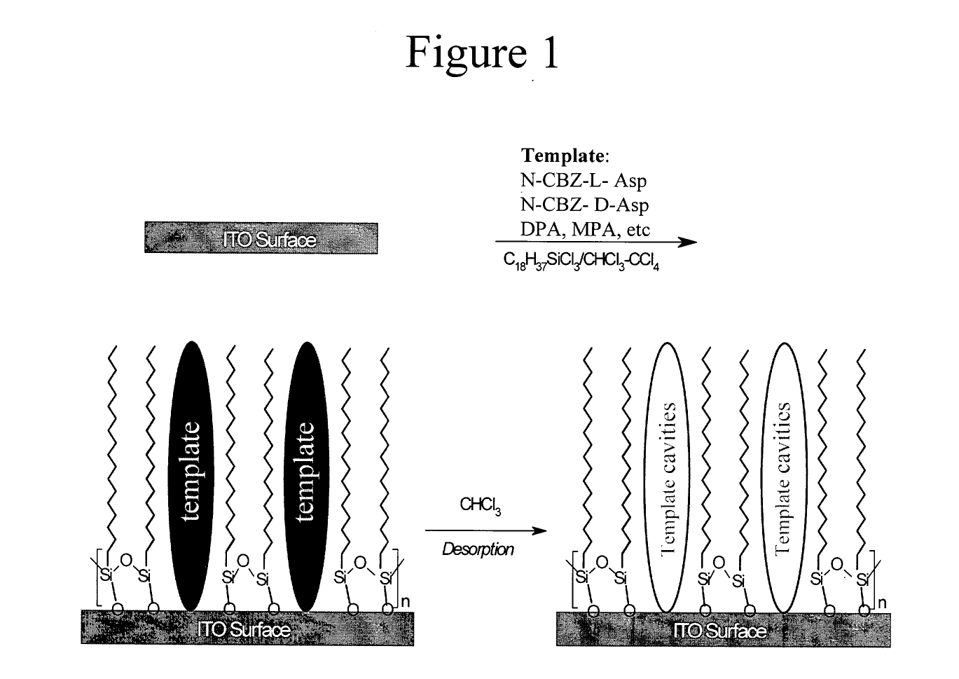

Sensor design using surface molecular imprinting technology (See FIG. 1) involves coating a support surface, such as an indium-tin oxide (ITO) glass electrode for example, with a polymer and embedding template molecules within the polymer layer. Polymer and template molecules may be co-adsorbed on a support (e.g., an electrode) surface by soaking the support surface in a suspension containing template molecules and polymer monomers. Support surfaces may include electrodes, optic fibers, polymer films, metal foil, semiconductors, quartz, glass, and ceramics. After being co-adsorbed on the surface, further polymerization in the presence of the template molecules may occur.

Template molecules may be removed from the polymer layer to provide size-, geometry-, and functionality-specific cavities for target molecules in solution. Since the template molecules are only physically adsorbed onto the support surface, they may be removed by solvent extraction, aging, heat treatment or neutral pH buffer, for example.

Without the template molecules embedded in the polymer layer, the electrode is coated with a polymer that contains cavities of specific size, geometry, and functionality according to the template molecule with which they were formed. Mainly, target molecules with size, geometry, and functionality complementary to the cavities may fit in the polymer's cavities and interact with the electrode surface. Thus, the template molecules used during imprinting are the same types of molecules that the sensor is created to detect.

§ 4.1.2 Fabrication of Exemplary Surface-Molecularly Imprinted Sensors for N-CBZ-ASP, MPA and DPA

An ITO-covered glass plate was used as the support surface to fabricate a surface-molecularly imprinted sensor for chiral molecules of N-CBZ-Asp, MPA and DPA. The glass plate was pretreated by a method described by Sagiv. (See, e.g., J. Sagiv, J. Am. Chem. Soc., 69, 2682 (1997).) Soaking the polar solid surface of the ITO glass plate (effective surface area about 1×4 cm²) in a suspension of N-CBZ-L-Asp, N-CBZ-D-Asp, DPA or MPA (templates) and octadecyltrichlorosilane (OTS, C₁₈H₃₇SiCl₃) (silylating agent) in CHCl₃/CCl₄ solution (2:3 v/v) at 0±1° C. for a period of time caused the polymer and template molecules to be co-adsorbed on the support surface. Thus, a hydrophobic monolayer of the polymerized organosiloxane groups was formed in the presence of the templates molecules. (See, e.g., J. Sagiv, J. Am. Chem. Soc., 102, 92 (1980).) The template molecules may be removed, for example, by rinsing the electrode with CHCl₃ (3×10 mL). The resulting sensor may then be dried by allowing it to stand overnight at room temperature.

Since OTS monolayers are stable in CHCl₃ and other solvents of low polarity, (See, e.g., J. Sagiv, Israel J. Chem., 18, 346 (1979), removing templates by repeated extractions with CHCl₃ creates the chiral cavities in a stable network of silane molecules as proposed by Sagiv et al. (See, e.g., J. Sagiv, J. Am. Chem. Soc., 102, 92 (1980).)

To find the optimal recognition conditions, the influences of OTS and templates concentrations in the deposition solution during sensor fabrication were examined, as shown in Table 1. Potential output responses were also studied as a function of adsorption time. After immersing pretreated ITO plates in a solution containing OTS and templates for durations varying from 1 to 10 minutes, the potential responses of these electrodes were shown in Table 1.

TABLE 1
Optimal condition of OTS and templates
concentration and adsorption time

| | CBZ-L-ASP | MPA | DPA |
|---|---|---|---|
| Adsorption time | 2-4 | 1-5 | 1-4 |
| (min) | | | |
| Conc. of | 0.0188- | 0.0195- | 0.0302-0.0405 |
| template (M) | 0.0376 | 0.0348 | |
| OTS (M) | 8 × 10⁻⁴ | 8 × 10⁻⁴ | 8 × 10⁻⁴ |

4.2 Theory and Operation of Surface-Molecularly Imprinted Sensors for Charged Species

The present invention couples surface molecular imprinting technology with electrochemical transduction to detect ionic molecules in solution. Surface imprinting technology gives sensors selectivity by creating a cavity with specific geometric features into which only molecules with complementary geometry may fit, as described in § 4.1.1.

Surface-molecularly imprinted sensors may be coupled with an electrochemical detection system to generate an output signal associated with recognizing a target molecule.

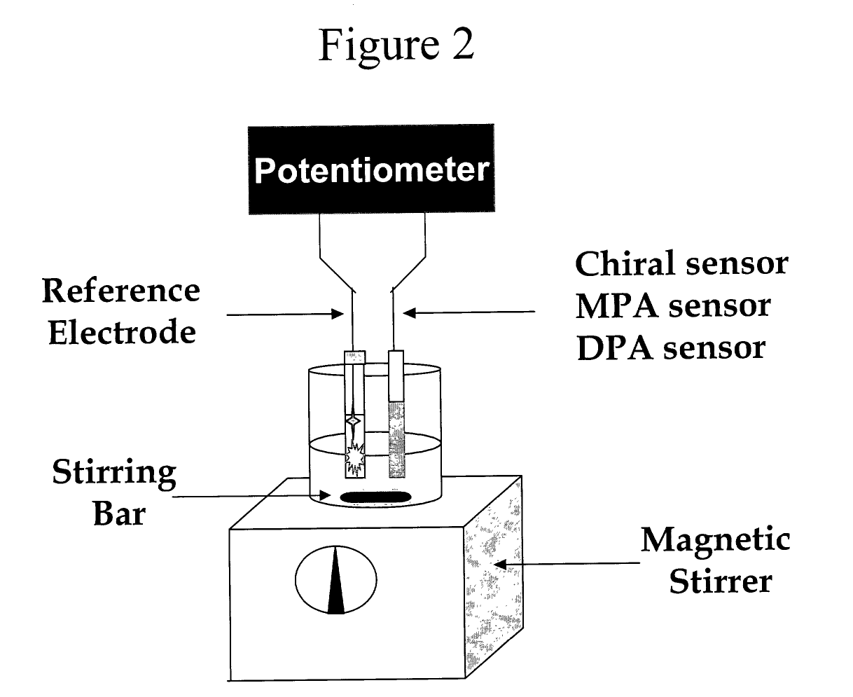

The electrochemical detection system may employ potentiometry, a technique that identifies specific analytes in solution by measuring the potential of reactions of interest in which those analytes are involved. FIG. 2 depicts an experimental assembly of a surface-molecularly imprinted sensor coupled with a potentiometer. The surface-molecularly imprinted sensor and a reference electrode may be immersed in a solution containing target molecules and other competing molecules. The electrodes are coupled to a potentiometer that measures the potential of reactions of interest occurring in the solution. Thus, the potential response of the sensor is defined as the difference between the electrode potential with and without the target molecule or the target molecule in solution, i.e., ΔE=E₁−E₀, where E₀ and E₁ are the electrode potentials before and after adding the target molecules, respectively.

In the present invention, the reaction of interest may be an electrostatic interaction between the target molecule in solution and the electrode surface. Such ionic interaction between the functionalized surface of the electrode and the target molecule provides the sensor's sensitivity and the mechanism that recognizes target molecules. For example, the electrostatic interaction may be a proton transfer between a proton-containing molecule in solution to the surface of a proton-sensitive electrode, such as an ITO electrode.

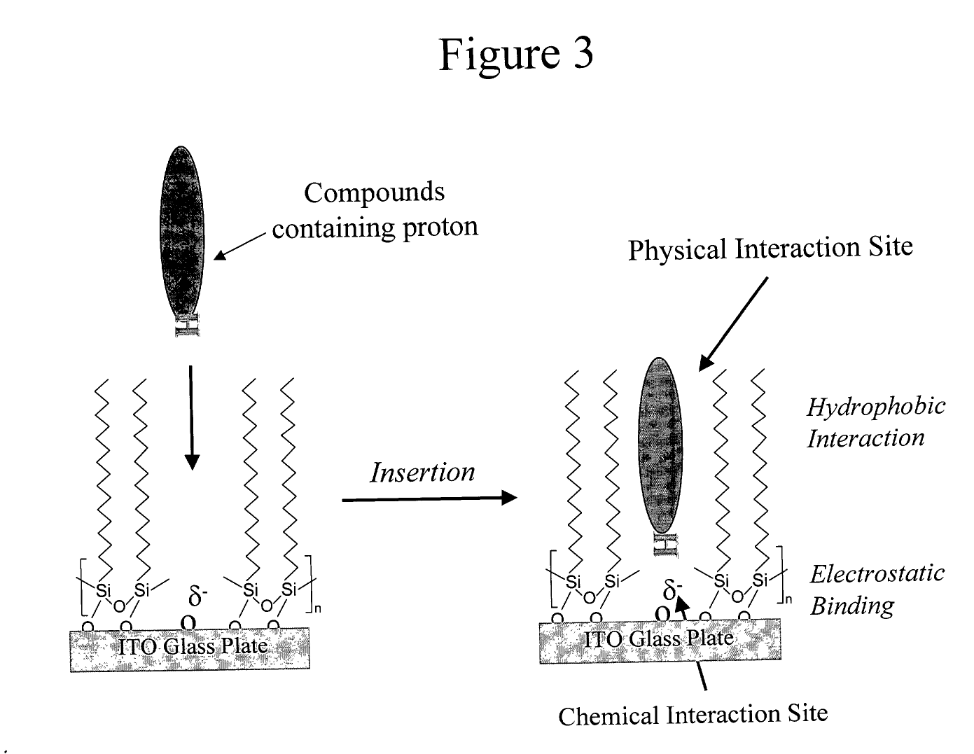

The reaction mechanism by which target molecules are detected may be divided into two parts: the hydrophobic interaction with the polymer monolayer (physical recognition); and the electrostatic binding with the electrode surface (chemical recognition). (See FIG. 3.) The hydrophobic interaction gives the sensor its selectivity since principally molecules with the same geometrical properties as the template molecules succeed in penetrating the polymer layer. The electrostatic interaction increases the chemical interaction energy between the target molecules and the electrode surface and allows the electrode surface to translate the recognition into a sensing event since the electrostatic binding changes the potential of the electrode surface.

In an exemplary embodiment of the present invention, ITO is used as the electrode, providing surface oxides with slightly negative charges. Proton in the target molecule has slightly positive charge of the target molecule's hydrogen atom. The oppositely charged atoms on the target molecule and the surface of the electrode facilitate electrostatic binding and provide a reaction of interest that may be recognized by potentiometry and translated into an output signal.

Strict limitations are not imposed on the types of molecules capable of being detected by surface-molecularly imprinted sensors. However, template molecules used for surface imprinting and their respective target molecules that may be detected by surface-molecularly imprinted electrochemical sensors should be molecules capable of electrostatic interaction with the electrode surface.

§ 4.3 Exemplary Embodiments

In the following, a surface-molecularly imprinted sensor for the chiral molecule N-CBZ-Asp is described in § 4.3.1. Next, a surface-molecularly imprinted sensor for dipicolinic acid is described in § 4.3.2. In § 4.3.3, a surface-molecularly imprinted sensor for methylphosphonic acid is described.

§ 4.3.1 Sensor for Chiral Molecules

The ability to detect chiral amino acids is important because they are essential substances in protein metabolism and in pharmaceutical and food products. (See, e.g., H. Bruckner, R. Wittner, M. Hausch, H. Godel, Fresenius Z. Anal. Chem., 333, 775 (1989).) As noted earlier, however, discriminating between enantiomers has been a challenge in the design of electrochemical sensors.

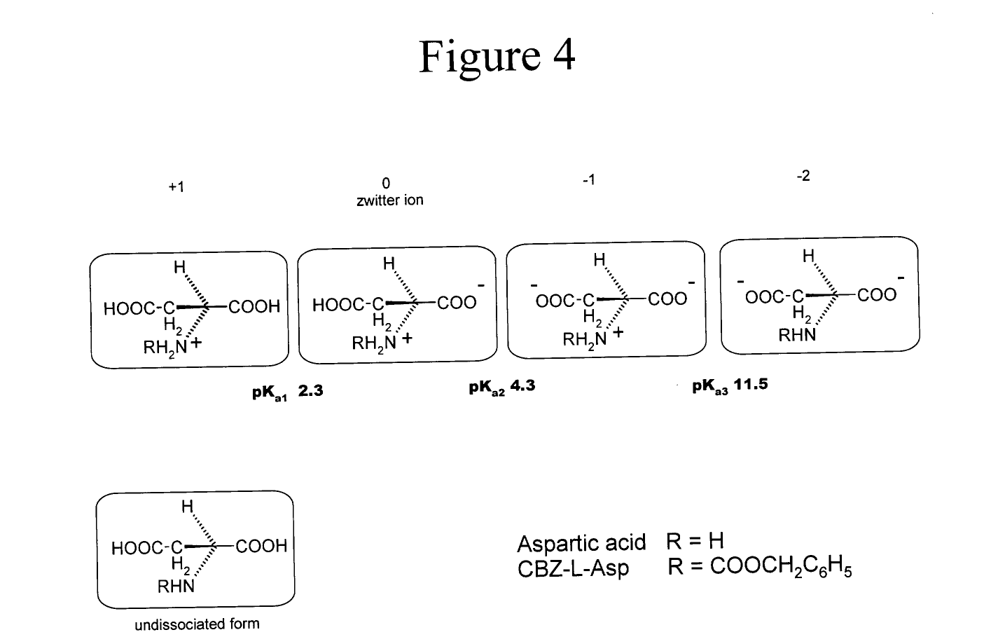

Cavities for surface-molecularly imprinted sensors for N-CBZ-Asp isomers may be fabricated for non-ionic, undissociated N-CBZ-Asp [N-CBZ-NHCH(COOH) (CH₂COOH)] since it may be dissolved in a low polarity medium (CHCl₃/CCl₄) during the adsorption step. Thus, in one embodiment, the sensors were designed not to accept zwitter ions and other ionic molecules. (See FIG. 4.) Surface molecularly imprinting previously had not been used to recognize ionic molecules because studies had shown that an OTS layer inserts only hydrophobic molecules. (See, e.g., J. Sagiv, J. Am. Chem. Soc., 102, 92 (1980), J. Sagiv, Israel J. Chem. 18, 346 (1979), J. Sagiv, Isr. J. Chem. 18, 339 (1979), D. F. Untereker, J. C. Lennox, L. M. Weir, P. R. Moses, R. W. Murray, J. Electroanal. Chem., 81, 309 (1977). The present invention expands the use of surface imprinting by applying it to detecting ionic molecules. In this exemplary embodiment of the present invention, surface-molecularly imprinted sensors had cavities formed in the OTS monolayer that were complementary to undissociated N-CBZ-L-Asp or N-CBZ-D-Asp. Electrode surfaces coated with imprinted OTS monolayers exhibited structural features related to specific geometries of the template molecules. (See, e.g., J. Sagiv, J. Am. Chem. Soc., 102, 92 (1980), J. Sagiv, Israel J. Chem. 18, 346 (1979), J. Sagiv, Isr. J. Chem. 18, 339 (1979).) Due to their complementary shapes, principally molecules with the same geometrical properties as the template molecules could enter the cavity, allowing the sensors' selectivity between the D- and L-isomers of N-CBZ-Asp to be studied by potentiometric measurements. The atomic charge densities on the nitrogen atom of N-CBZ-L-Asp, calculated by the semiempirical MO (molecular orbital) method (PM3)(Stewart), are significantly different for the undissociated and dissociated molecules (−0.02, +0.453 and +0.505 for the undissociated aspartic acid, zwitter ion and cation, respectively), indicating a considerable increase in polarity for the ionic species. The present invention exploits the polarity of the target molecules by using a combination of electrostatic interaction (with the electrode surface) and hydrophobic interaction (with the OTS monolayer) to recognize N-CBZ-Asp.

More specifically, the present invention uses long-range electrostatic energy interactions to recognize target molecules. Thus, the target molecules in solution should be electrolytic. (See, e.g., C. Mavroyannis, M. Stephen, J. Molec. Phys., 5, 629 (1962).) In this embodiment of the present invention, surface-molecularly imprinted sensors were fabricated according to the method described in § 4.1.2 above for chiral recognition of D- or L-isomers of N-CBZ-Asp. The imprinted sensor surface works like a channel gate that opens only for the D- or L-isomer according to the template used. Co-adsorbing OTS with D- or L-Asp during hydrolysis creates a D- or L-imprinted monolayer, and only those N-CBZ-Asp molecules of proper chirality can “carry” a proton to the ITO surface. Thus, the OTS sensor responds to pH, which changes when the L- or D-amino acid is inserted into the OTS monolayer. Control electrodes were also fabricated by modifying an ITO electrode with OTS without N-CBZ-Asp cavities.

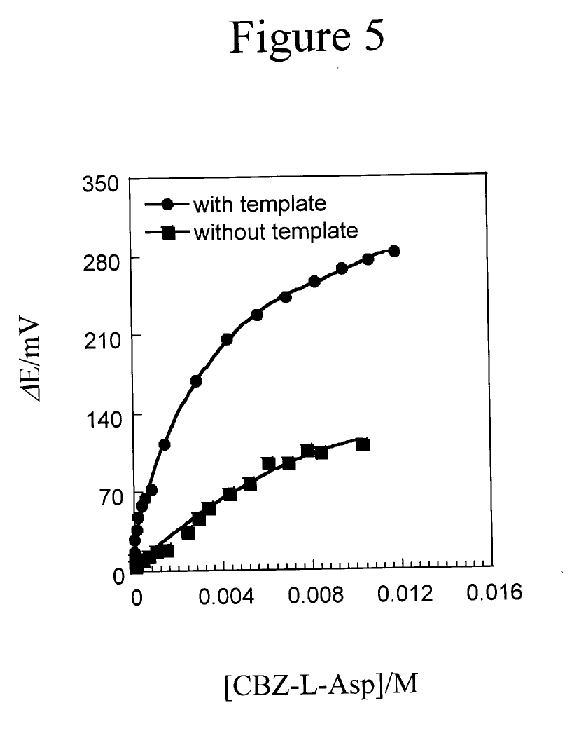

To ensure that the surface-molecularly imprinted sensor for the chiral amino acid N-CBZ-L-Asp contained cavities specific for its target molecules, N-CBZ-L-Asp was detected by the surface-molecularly imprinted sensor and a control electrode. As shown in FIG. 5, the surface-molecularly imprinted sensor for N-CBZ-L-Asp had greater potential responses than the electrode coated with OTS without N-CBZ-L-Asp cavities, indicating that the surface-molecularly imprinted sensor recognizes this chiral compound. The target molecule was 1500 times more concentrated on the surface-molecularly imprinted sensor for N-CBZ-L-Asp than the electrode coated with OTS without cavities. Thus, N-CBZ-L-Asp was initially incorporated into the adsorbed OTS monolayer during sensor fabrication and subsequently extracted to create the chiral recognition cavity. Incorporating N-CBZ-Asp into the polysiloxane film followed by its departure from the monolayer also was confirmed by X-ray photoelectron spectroscopy (using Quantum 2000 (pH 1 Co)). The completed surface-molecularly imprinted sensor's nitrogen and oxygen intensities were restored to their initial values (<±5% error) (e.g., prior to imprinting) after L- or D-Asp was removed from the monolayer, as shown below in Table 2.

TABLE 2
Elemental analysis results

| Element | C | O | In* | Sn | N | Si |
|---|---|---|---|---|---|---|
| ITO glass plate | 186 | 189 | 100 | 6.6 | 7.9 | 7.65 |
| Adsorbed CBZ-Asp | 396.97 | 219.7 | 100 | 7.05 | 17.2 | 16.29 |
| and ODS on ITO | | | | | | |
| glass plate | | | | | | |
| Rinsed | 351.33 | 191.33 | 100 | 7.87 | 6.13 | 10.07 |

*Normalized to Indium.

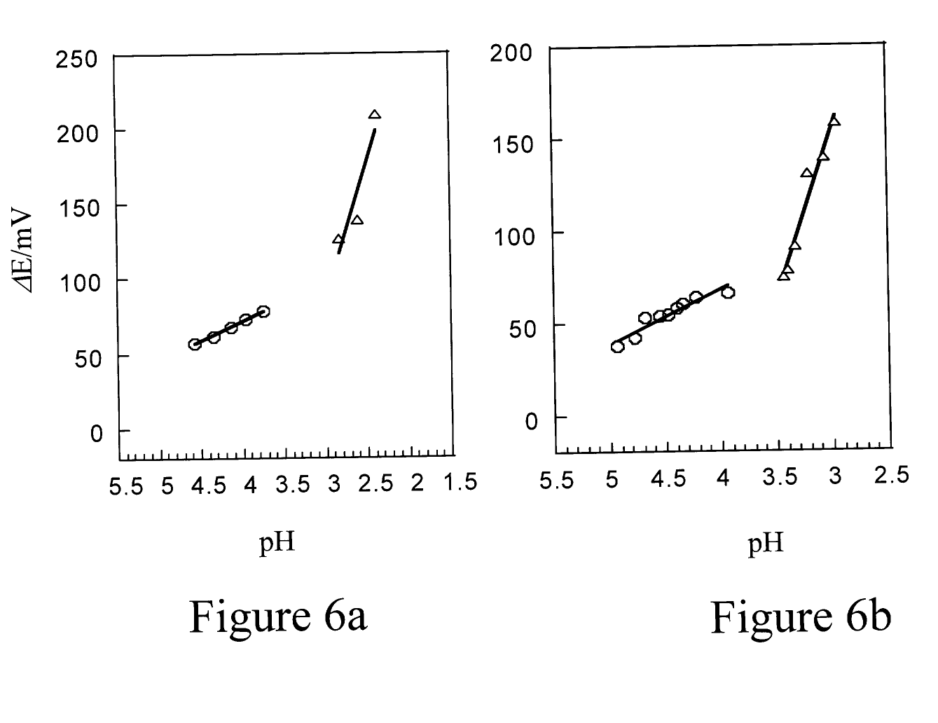

As seen in Table 2, the ratio of CBZ-Asp to OTS on the ITO surface during co-adsorption is 1:2.38. Thus, 29.4% of the material co-adsorbed on the electrode's surface was N-CBZ-Asp, which was washed away after solvent extraction. As previously mentioned, the pH of N-CBZ-Asp solutions was expected to have an influence on detecting aspartic acid isomers because of the three acid/base equilibria of N-CBZ-Asp. (See FIG. 4.) Dissociation constants (pKₐ) for N-CBZ-Asp were obtained by titration on a pH meter. An experimental assembly similar to the one described in § 4.2, but having an additional electrode for measuring pH changes, detected potential responses of the surface-molecularly imprinted sensors for N-CBZ-Asp with and without the target amino acids in solution as a function of pH. FIGS. 6a and 6b show the results of testing the potentiometric response of the N-CBZ-L-Asp sensor (b) and the ITO electrode (a), respectively, towards protons. Both the N-CBZ-Asp sensor and the ITO electrode exhibit a two-region voltage-pH response. The combined overall slope of voltage vs. pH indicated that the pH sensitivities of the sensor and ITO electrode are unusually high compared to other electrodes, which exhibit slopes of 59 mV dec⁻¹. Since the unmodified ITO electrode responded similarly to the surface-molecularly imprinted sensor for N-CBZ-Asp, the extraordinary responses should be attributed to ITO itself.

Discontinuity in the electrodes' potential responses as a function of pH (See FIGS. 6a and 6b.) may be attributed to the structure of the ITO electrodes. ITO is a degenerate n-type semi-conducting material with vertical column growth in multiple orientations. (See, e.g., A. K. Kulkarni, K. H. Schulz, T. S. Lim, M. Khan, Thin Sold Films, 308-309, 1 (1997).) Individual columns are single crystals with grain sizes ranging from a few nanometers to tens of nanometers. Below a certain size scale, the electrochemical properties of nanostructured electrodes have been shown to alter discontinuously. (See, e.g., Electrochemistry of nanomaterials, Hodes, G. Eds., VILEY-VCH Verlag GmbH: Weinheim, (2001).) This scale-sensitive phenomenon may explain the observation that both the bare ITO and N-CBZ-L-Asp sensor responded to protons with discontinued slopes at different pH ranges and showed unusually steep slopes.

As described in § 4.2, the interaction between the amino acid and surface-molecularly imprinted sensor includes a hydrophobic interaction with the OTS monolayer (which provides selectivity) and an electrostatic binding with the surface oxides (which provides sensitivity). (See FIG. 3.) The electrostatic binding changes the potential of the ITO glass plate, thereby allowing the sensor to specifically recognize the target molecules. Since the OTS monolayer can accommodate only a neutral amino acid, the ITO surface plays an important role in the recognition process. TiO₂ (See, e.g., U. Weimar; Gopel W. Sensors and Actuators B, 26-27, 13 (1995).) and SnO₂ (See, e.g., G. J. Li, S. Kawi, Talanta, 45, 759 (1998).), have high selectivities and sensitivities to H₂ gas. As shown in FIG. 6a, the ITO electrode also has a higher sensitivity to protons (H⁺). After N-CBZ-L-Asp molecules were inserted into the OTS monolayer without dissociation, the ITO could still sense a proton (See FIG. 3).

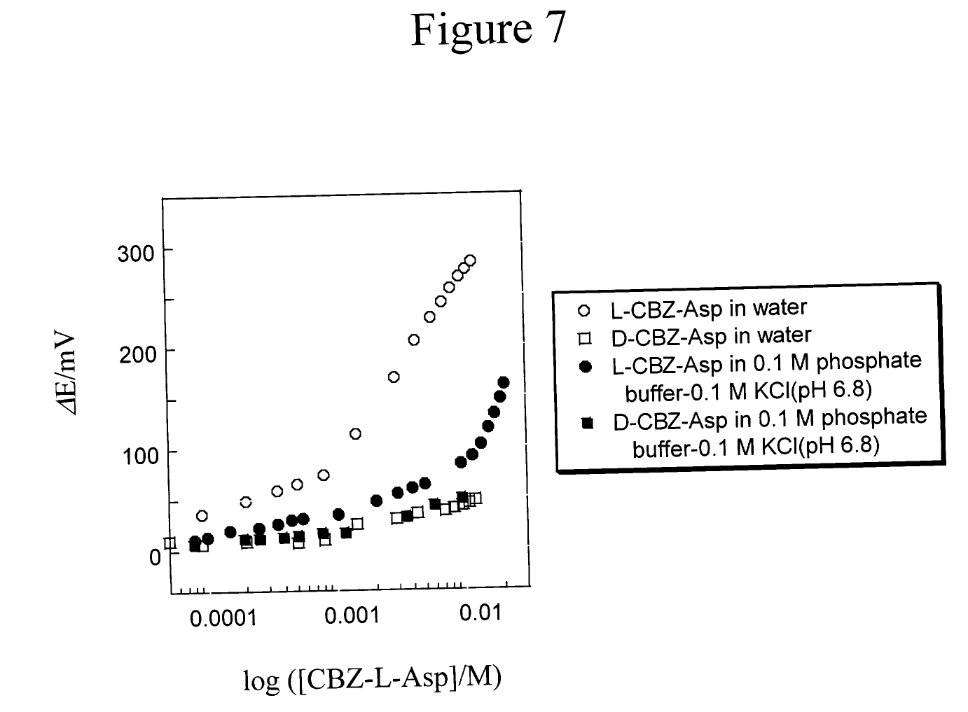

FIG. 7 shows the calibration curves for the isomers of N-CBZ-Asp dissolved in water (◯, □) and in phosphate buffer-0.1 M KCl (●, ▪) at pH 6.8. The greater response for the L-isomer in both cases indicates that the surface imprinting technique was effective in creating a sensor with selectivity towards one isomer. ΔE for the L-isomer dissolved in water (◯) was proportional to the logarithmic concentration of N-CBZ-L-Asp with a slope of about 30 mV dec⁻¹ in a concentration range of 5×10⁻⁶ to 8×10⁻³ M and 180 mV dec⁻¹ over 1.4×10⁻³ to 1.18×10⁻² M, while the slope for the D-isomer was about ˜0 mV dec⁻¹. Therefore, the surface-molecularly imprinted sensor for N-CBZ-L-Asp was chiral-selective with respect to the L-isomers, indicating that the D-isomer was much more restricted from interacting with the imprinted OTS monolayer than the L-isomer. Thus, the inventors believe the L-isomer participated in forming impressions in the OTS monolayer during sensor fabrication. The L-isomer created adsorption sites in the subsequent adsorption step that were characterized by its three-dimensional geometry. (See, e.g., J. Sagiv, J. Am. Chem. Soc., 102, 92 (1980), J. Sagiv, Israel J. Chem. 18, 346 (1979), J. Sagiv, Isr. J. Chem. 18, 339 (1979).) Consequently, N-CBZ-Asp molecules of proper chirality can “carry” a proton to the electrode's ITO surface.

An additional interesting discovery is seen in FIG. 7. The potentiometric selectivity coefficient for N-CBZ-L-Asp with respect to N-CBZ-D-Asp,

$${K}_{LD}^{POT} ,$$

is 0.08. This value is 10 times lower than

$${K}_{LD}^{POT}$$

found in water (9×10⁻³) , as is shown in the calibration curves for the enantiomers of N-CBZ-Asp dissolved in phosphate buffer-0.1 KCl (●,▪). Two reasons may explain this phenomenon. First, the OTS sensor responds to the protons of N-CBZ-Asp. If this is the case, the buffer solution would not control the pH of the solution. Another reason may be that amino acids are self-electrolyte analytes (See, e.g., J. Stenesh, Biochemistry, Plenum Press, New York, p16 (1998), C. Mavroyannis, M. Stephen, J. Molec. Phys., 5, 629 (1962).), since they are both acids and amines. If this is the case, the amino acids themselves could act as the supporting electrolyte.

Furthermore, K₂HPO₄, KOH and KCl, which were used to prepare the phosphate buffer-KCl solution, are smaller molecules than the amino acids. If these smaller molecules were to occupy the cavities, the amino acids would be blocked from entering such cavities, resulting in decreased detection efficiency. The smaller potential responses that were obtained when pH was regulated with a buffer also supports this mechanism hypothesis (See FIG. 7.). Therefore, the supporting electrolyte-buffer solution, which is usually used for the control of the migration of the species and the pH of the solution, was replaced by water during experimentation.

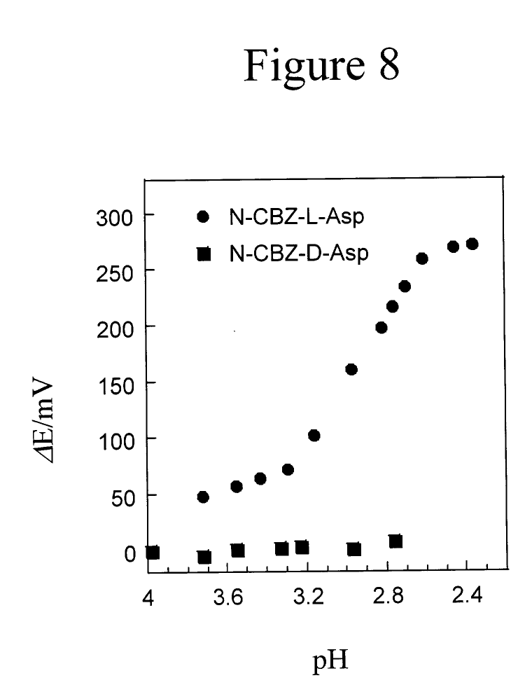

To delineate the recognition mechanism, the pH dependence of the sensor output was measured (See FIG. 8.) by using the data from FIGS. 5 and 7. Decreases in pH were coupled with increases in the N-CBZ-L-Asp concentration, showing that the sensor responds to protons and that potential changes arose only from the N-CBZ-Asp molecules since amino acids were the only electrolytes added to the solution.

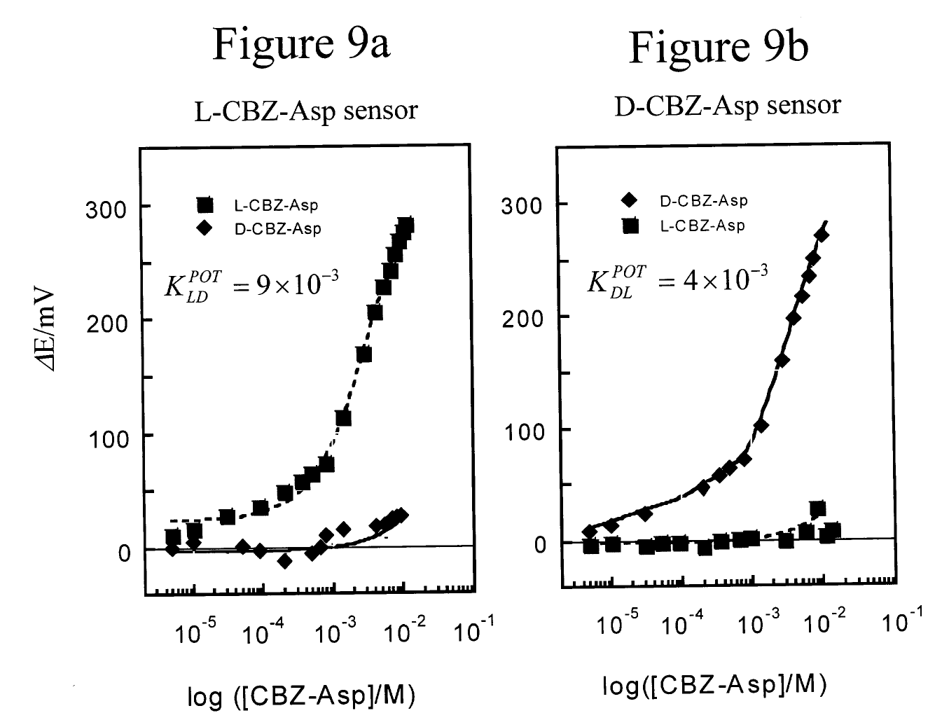

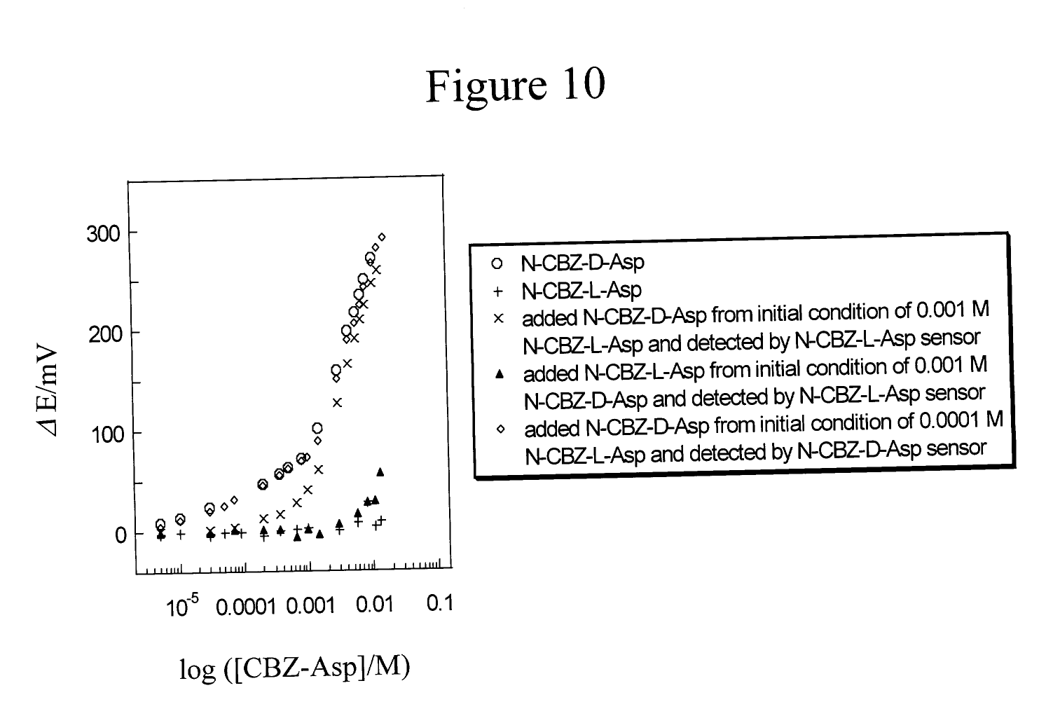

The surface-molecularly imprinted sensors for L- or D-aspartic acid isomers demonstrated selective recognition ability in the presence of the other isomer, as shown in FIGS. 9 and 10. FIG. 9 shows that surface-molecularly imprinted sensors for N-CBZ-L-Asp and N-CBZ-D-Asp recognized the L-and D-isomers of N-CBZ-Asp, respectively. FIG. 10 shows that the surface-molecularly imprinted N-CBZ-D-Asp sensor selectively recognized the D-isomer even when starting conditions contained some concentration of the L-isomer. Table 3 (below) shows that each surface-molecularly imprinted sensor gave high responses towards only one of the N-CBZ-Asp isomers in racemic solutions.

TABLE 3
Output of sensors for racemic samples (50% CBZ-L-Asp and 50% CBZ-D-Asp)

| | D-Asp sensor output/Mv | | | L-Asp sensor output/mV | | |
|---|---|---|---|---|---|---|
| | 50% D- | | | 50% D-and | | |
| Concentration | and 50% | 100% | | 50% | 100% | |
| mM^{a} | L-isomers | D-isomer | Error % | L-isomers | L-isomer | Error % |
| 0.095 | | | | 38 | 37 | 2 |
| 0.20 | 46 | 47 | −2 | 49 | 49 | 0 |
| 0.33 | 55 | 55 | 0 | 60 | 59 | 2 |
| 0.50 | 62 | 62 | 0 | 67 | 65 | 3 |
| 0.64 | 67 | 68 | −2 | 75 | 72 | 4 |
| 0.79 | 72 | 71 | 2 | 79 | 78 | 1 |
| 0.93 | 76 | 76 | 0 | 84 | 84 | 0 |
| 1.49 | 100 | 100 | 0 | 116 | 114 | 2 |
| 2.79 | 165 | 158 | 4.5 | 174 | 172 | 1 |
| 4.09 | 191 | 192 | 0 | 210 | 205 | 2.5 |
| 5.32 | 203 | 203 | 0 | 234 | 222 | 5 |

^{a}Concentrations of CBZ-L- and/or -D-Asp

Results depicted potential increases of about 1% in the presence of equimolar amounts of the counter isomer. No substantial potential difference was observed with and without the counter isomer, demonstrating the high selectivity that was expected from the selectivity coefficients.

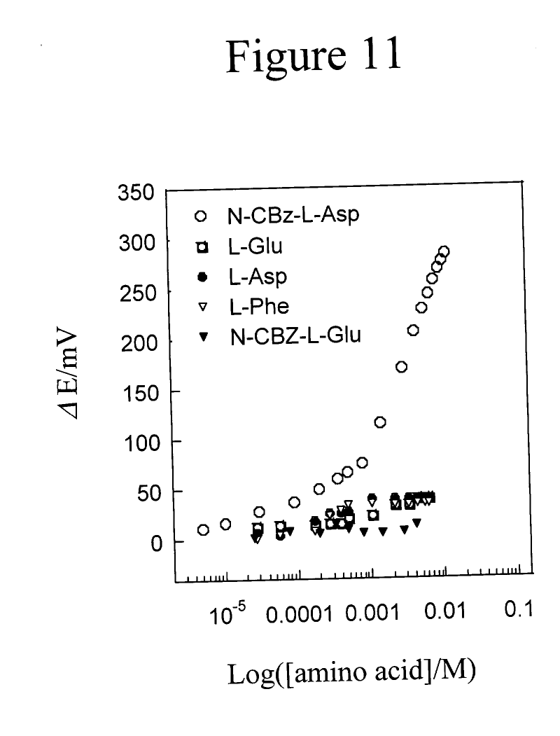

The surface-molecularly imprinted sensors had virtually no response towards other amino acids, even those that are structurally similar to N-CBZ-Asp. For example, N-CBZ-L-Glu (▾), L-Asp (●), L-Glu [symbol] and L-Phe (∇) (FIG. 11) exhibited slopes of ˜0 mV dec⁻¹ (K^{POT}_{N-CBZ-L-ASP, interfere amino acids}≈0.001) on the surface-molecularly imprinted sensor for N-CBZ-L-Asp. Although N-CBZ-L-Asp (HOOC—CH₂—CHNHR—COOH) and N-CBZ-L-Glu (HOOC—CH₂—CH₂—CHNHR—COOH) are structurally alike except for N-CBZ-L-Glu's additional CH₂ group, the N-CBZ-L-Asp sensor could distinguish between these amino acids.

The potentiometric selectivity coefficient for the L-isomer sensor was evaluated by the following equations:

$$\begin{matrix}{E}_{L} = {E}_{L}^{0} + s {log [ {H}^{+} ]}_{0} \\ = {E}_{L}^{0} + s log ( [ N - CBZ - L - Asp ] + {K}_{LD}^{POT} [ N - CBZ - D - Asp ] )\end{matrix} \qquad ( 1 )$$

where [H⁺]₀ is the proton concentration on the ITO surface; E_{L} and E_{L}⁰ are the potential of the sensor and the standard electrode potential, respectively; s is the slope as shown in FIG. 6; and

$${K}_{LD}^{POT}$$

is the selectivity coefficient for L-isomer in the presence of D-isomer. (See, e.g., C. M. A. Brett, A. M. O. Electroanalysis, Oxford Science Publications, Oxford University Press: Oxford, p40 (1998).) The concentration of N-CBZ-L-Asp was calculated using pKₐ₁=2.3 and pKₐ₂=4.3. Using s values that were evaluated from the first derivative of the pH response simulated the output potentials for the L- and D- sensors. From the simulation,

$${K}_{LD}^{POT}$$

and

$${K}_{LD}^{POT}$$

values of 9×10⁻³ and 4×10⁻³ were obtained for the L- and D-isomer sensors, respectively. These values indicate that the sensitivities of the sensors for their target isomer are 110-250 times greater than those for their counter isomers. Compared to previously reported molecularly imprinted polymer-film sensors (See, e.g., Molecular and Ionic Recognition with Imprinted Polymers, R. A. Bartsch, M. Maeda, Eds.; American Chemical Society: Washington D.C., (1998), D. Kritz, O. Ramstrom, A. Svensson, K. Mosbach, Anal. Chem., 67, 2142 (1995).), the present invention's surface-molecularly imprinted sensors have a remarkable performance with a selectivity coefficient of 4×10⁻³. The D-isomer sensor, for example, should produce a 5% output potential error if the D-isomer concentration is 20 times (30 mM) as high as the L-isomer concentration (1.5 mM).

The surface-molecularly imprinted sensor's response time, evaluated as the time needed for a 95% signal change, was about 160 s for 2.0×10⁻⁴ M N-CBZ-L-Asp. Average potential variations of 0.60% as a relative standard deviation were observed for 10 consecutive determinations of 10 mM N-CBZ-L-Asp. After measurements were repeated more than 200 times, the response still remained 92% of its initial magnitude, demonstrating the long lifetime of the sensors.

§ 4.3.2 Dipicolinic Acid Sensor

Rapid identification of Bacillus anthracis spores is important because of its potential use as a biological warfare agent. (See, e.g., W. Barnaby, The Plague Makers: The Secret World of Biological Warfare, Vision Paperbacks: London, (1997).) Considering emergency response plans for such an attack with biological weapons, developing an inexpensive, rapid, and sensitive field portable sensor is important.

Dipicolinic acid (pyridine-2, 6-dicarboxylic acid; DPA) is a major constituent of bacterial endospores (including Bacillus anthracis spores), comprising 5 to 14% of their dry weight. (See, e.g., W. G. Murrell, A. D. Warth, Spores III. American Society for Microbiology; L. L. Campbell, L. L. Halvorson, H. O. eds.; Ann Arbor: Mich. P. 1-24 (1965), A. D. Warth, Adv. Microb. Physiol., 17, 1-45 (1978).) Analysis of DPA is also important in studies dealing with sporulation, germination, and spore structure. Furthermore, the presence of DPA is considered diagnostic for bacterial endospores. Combining surface molecular imprinting technology with potentiometry may be used to fabricate a chemical sensor for dipicolinic acid.

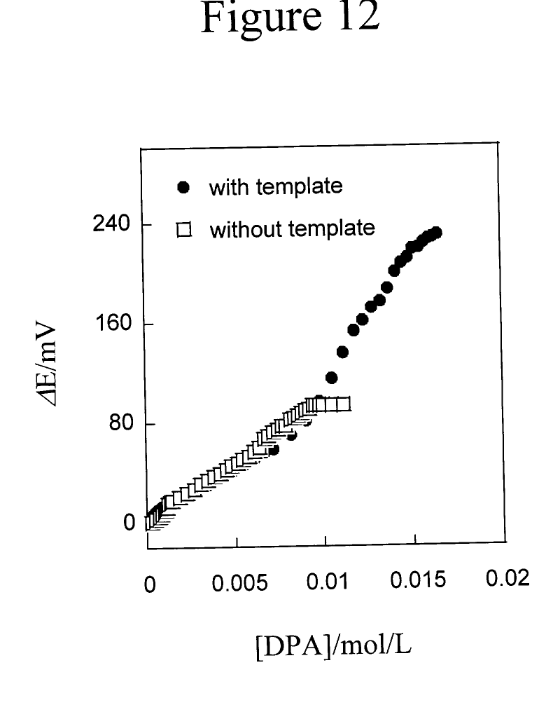

A surface-molecularly imprinted sensor for DPA was fabricated according to the method described in § 4.1.2 above. As shown in FIG. 12, the surface-molecularly imprinted electrode for DPA had greater potential responses to DPA in solution than a control electrode modified with OTS but lacking cavities for DPA. (All measurements for this exemplary embodiment were made in 50 ml of 0.1 M phosphate buffer saline (PBS) (pH 7.2), thermostated to 0° C., in a 100 mL working volume electrochemical cell, as shown in FIG. 2.) These results suggest that the surface of the surface-molecularly imprinted electrode for DPA exhibited structural adsorption properties related to specific geometrical features of the displaced molecules. Moreover, the DPA adsorbed in the sensor was concentrated by 23 times as much as that of control. Imprinting the OTS monolayer with DPA during hydrolysis created a DPA-imprinted monolayer, allowing DPA molecules to “carry” a proton to the ITO surface. The surface-molecularly imprinted sensor therefore responds to pH, which changes upon insertion of DPA into the OTS layer. Thus, the imprinted cavity works like a channel gate that opens for DPA molecules.

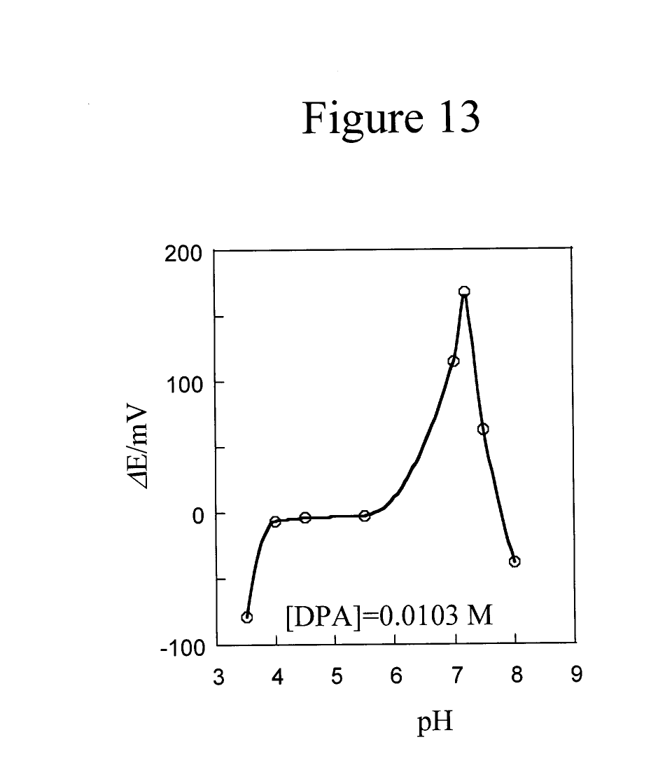

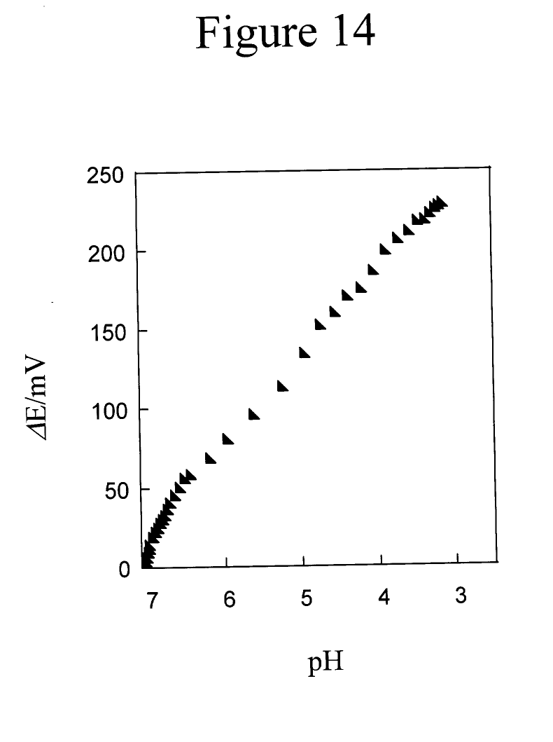

FIG. 13 shows the pH profile for the surface-molecularly imprinted sensor for DPA. The pH has a marked influence on the recognition process of this sensor for DPA, and the optimal pH was found at 7.2. FIG. 14 shows that as pH decreases, DPA concentration increases, indicating that the sensor responds to protons. This phenomenon may be explained by the function of the transducer, ITO, which interacts with the hydrophilic DPA in the presence of a hydrophobic monolayer. After DPA was inserted into the OTS monolayer, ITO was able to sense protons even if the DPA molecules were not dissociated, same principle as in § 4.3.1.

Using this model, the interaction between DPA and its surface-molecularly imprinted sensor can be divided into two parts: (i) hydrophobic interaction with the OTS monolayer created during the self-assembly process, and (ii) electrostatic binding with the electrode's surface oxides. (See FIG. 3.) The first interaction provides selectivity to the sensor since principally molecules with the same geometrical properties as the template molecules may penetrate the OTS monolayer. The second electrostatic interaction boosts the total chemical interaction energy into an increase in the affinity of the molecules for the site. After electrostatic binding, the proton-sensitive ITO surface translates the molecular recognition event to the surface-molecularly imprinted chemical sensor for DPA.

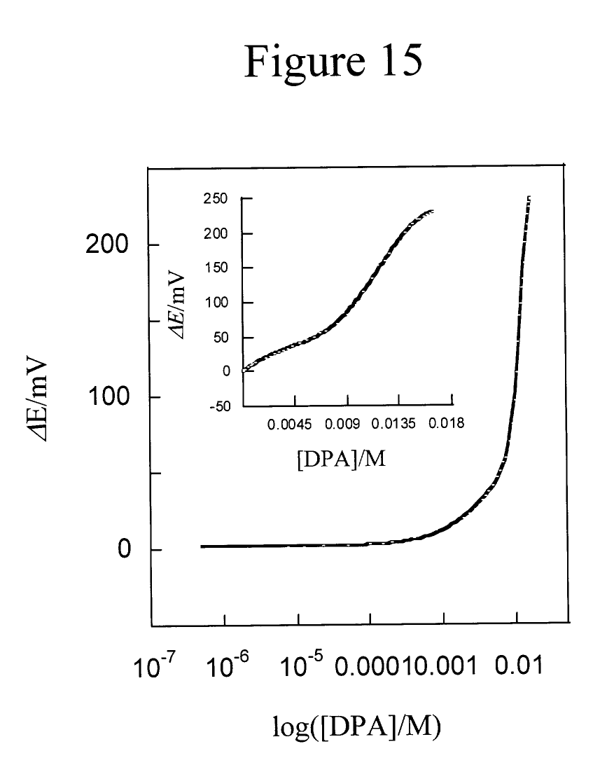

The calibration plot for DPA using the surface-molecularly imprinted sensor is shown in FIG. 15. The sensor's minimum detectable amount (MDA) of DPA was 1.5 μM and it had a linearity range of 1.5×10⁻⁶-0.01935 M. The surface-molecularly imprinted sensor for DPA had an operating range that varied within two orders of magnitude.

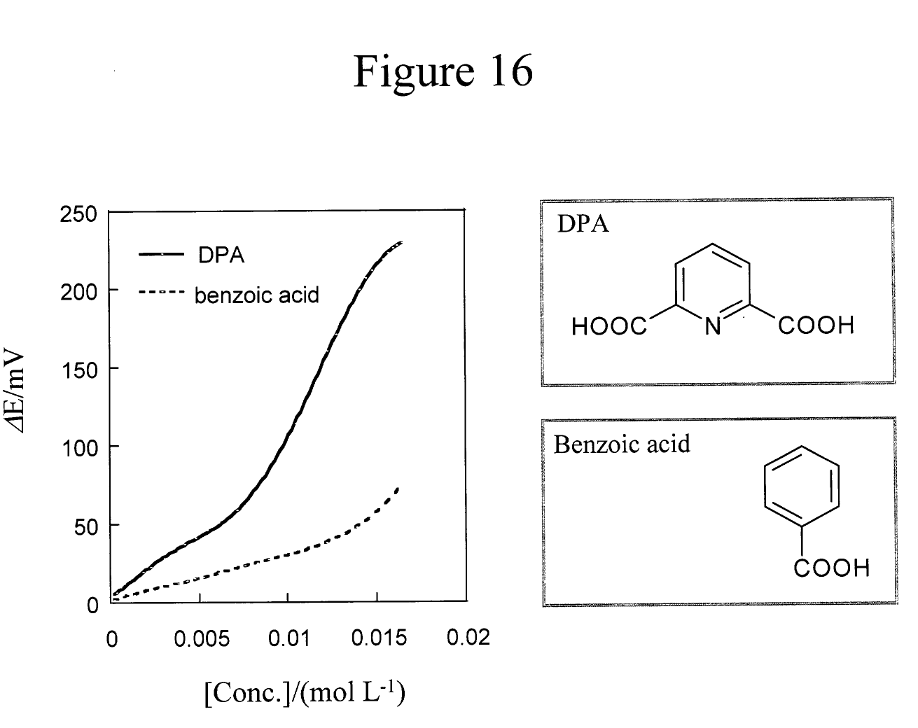

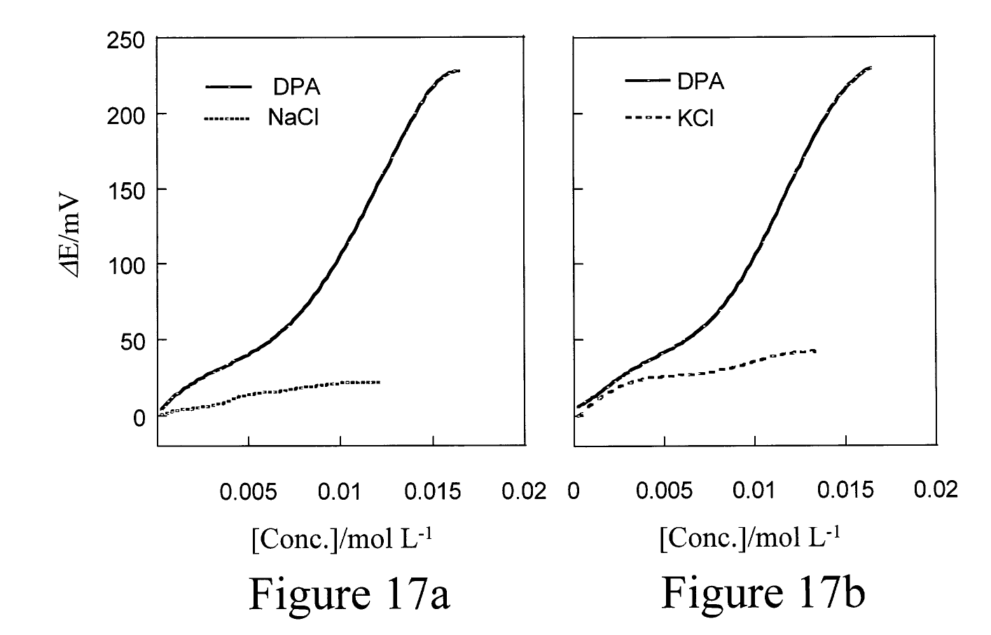

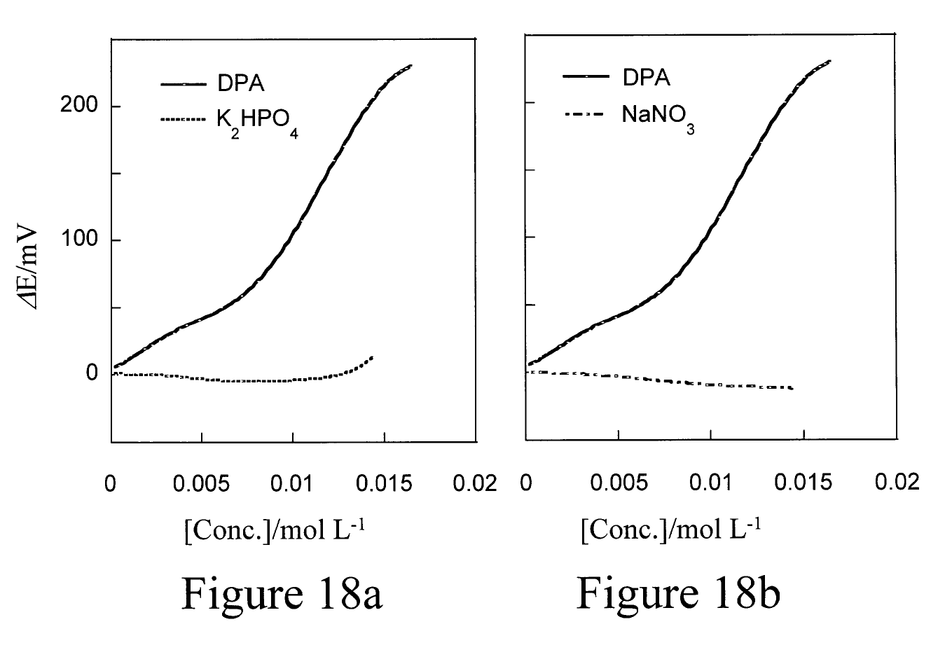

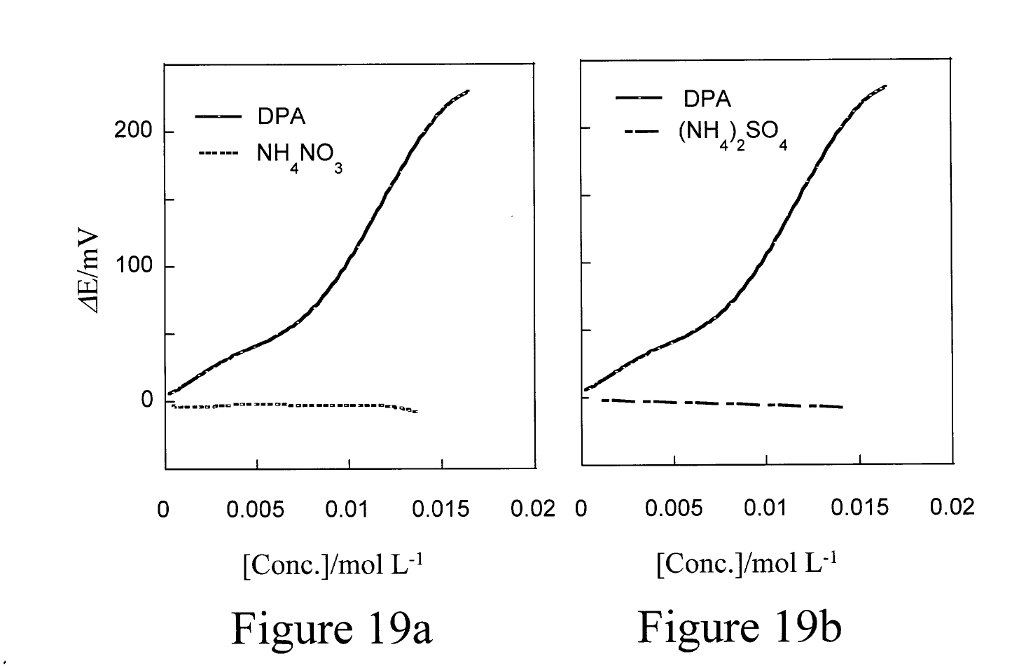

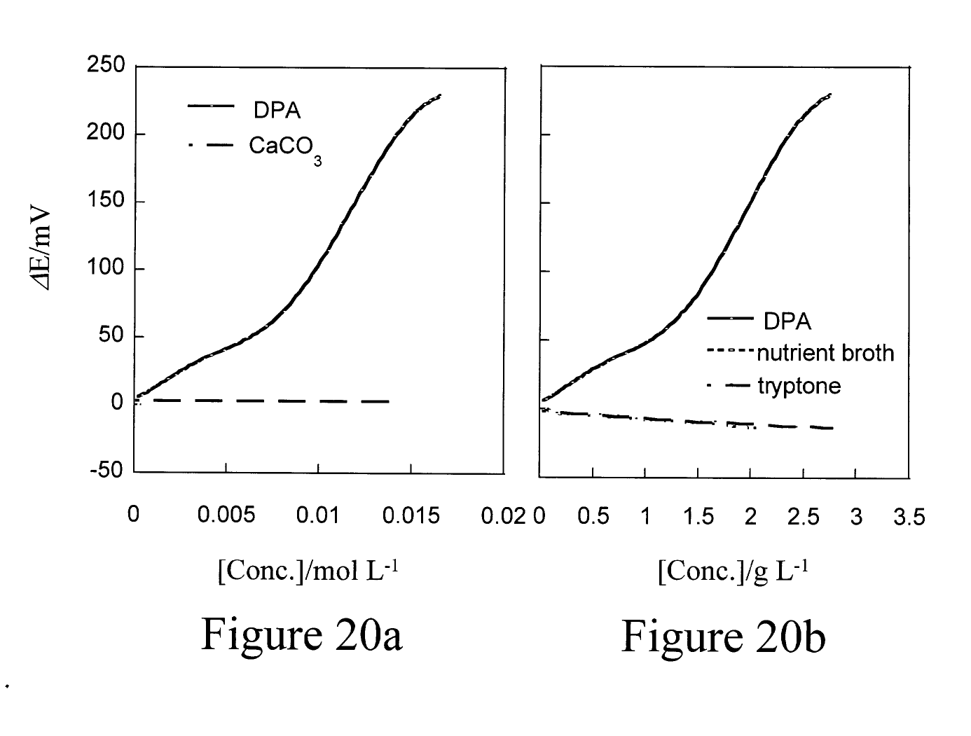

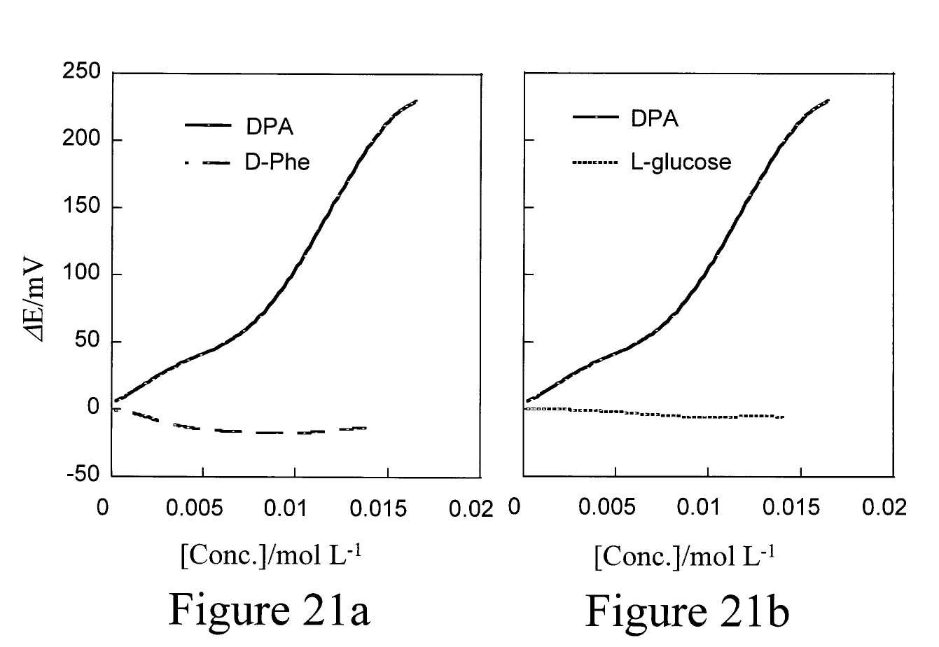

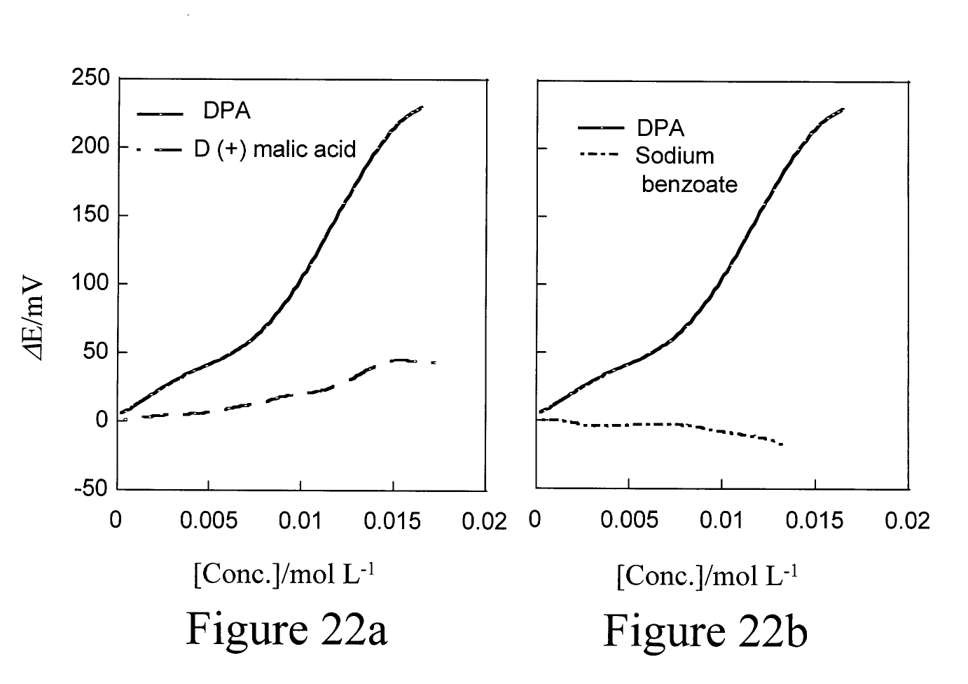

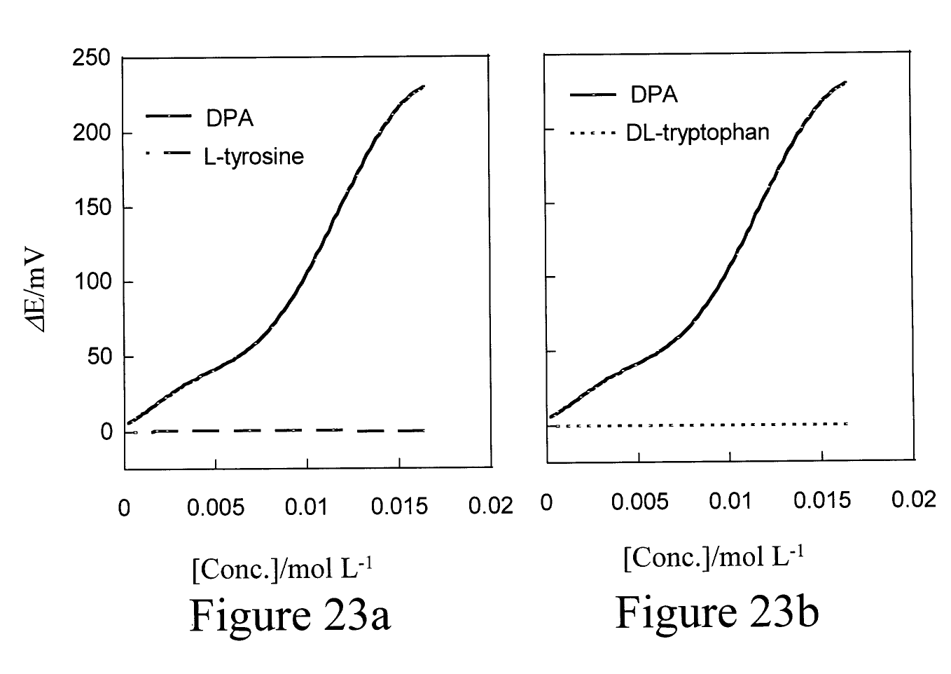

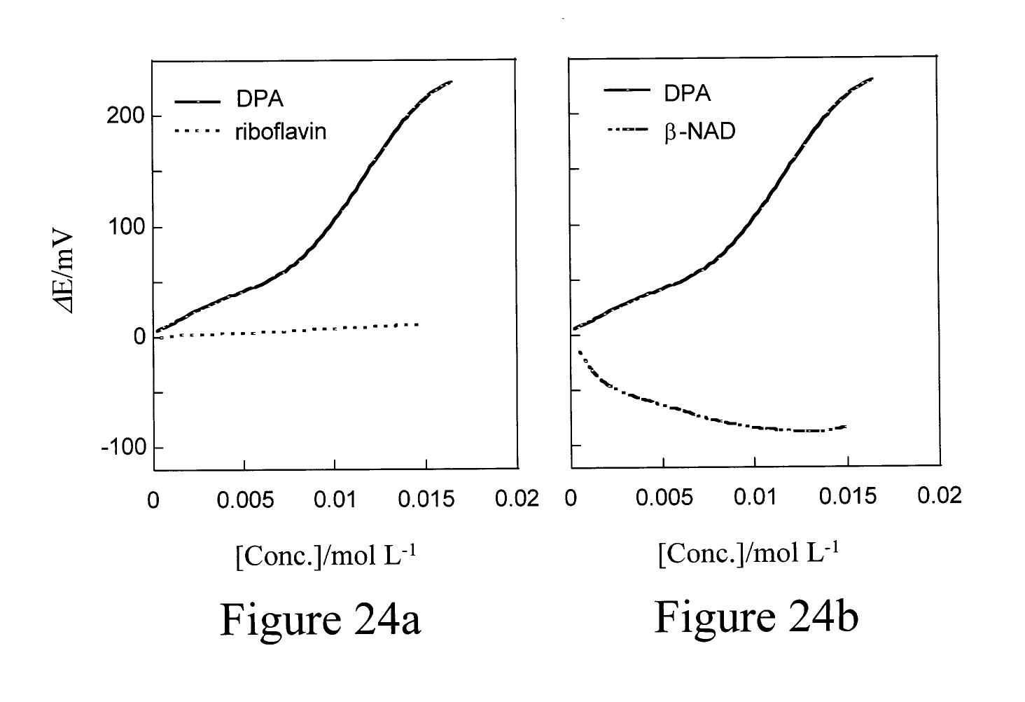

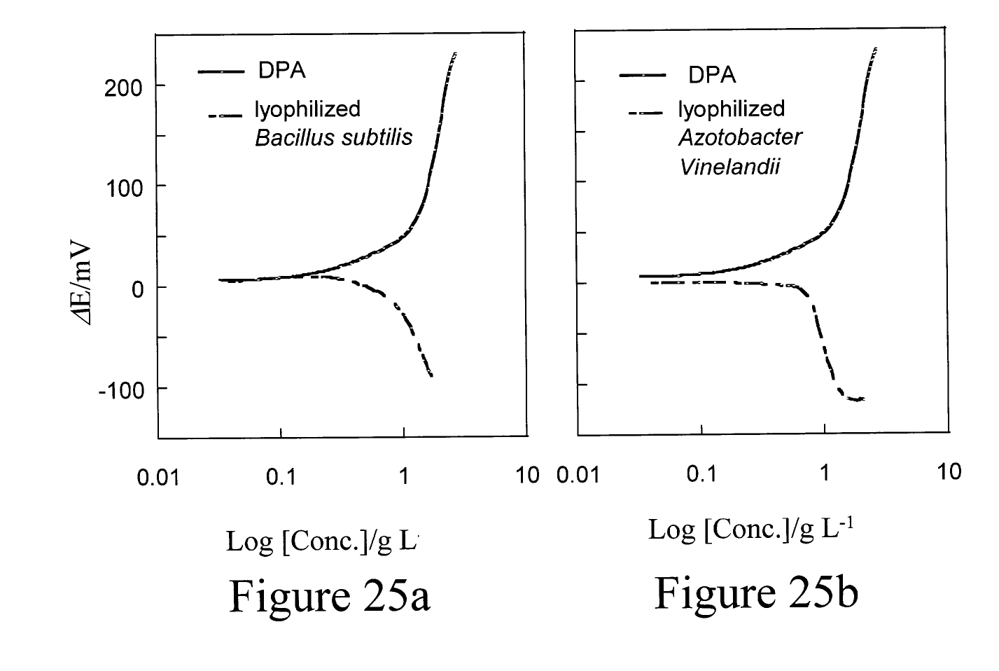

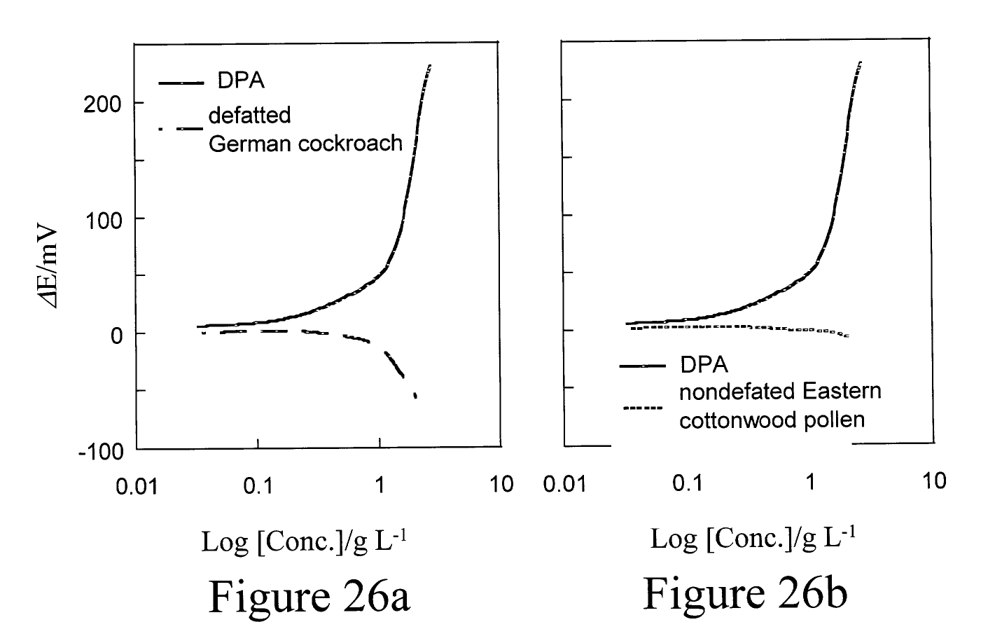

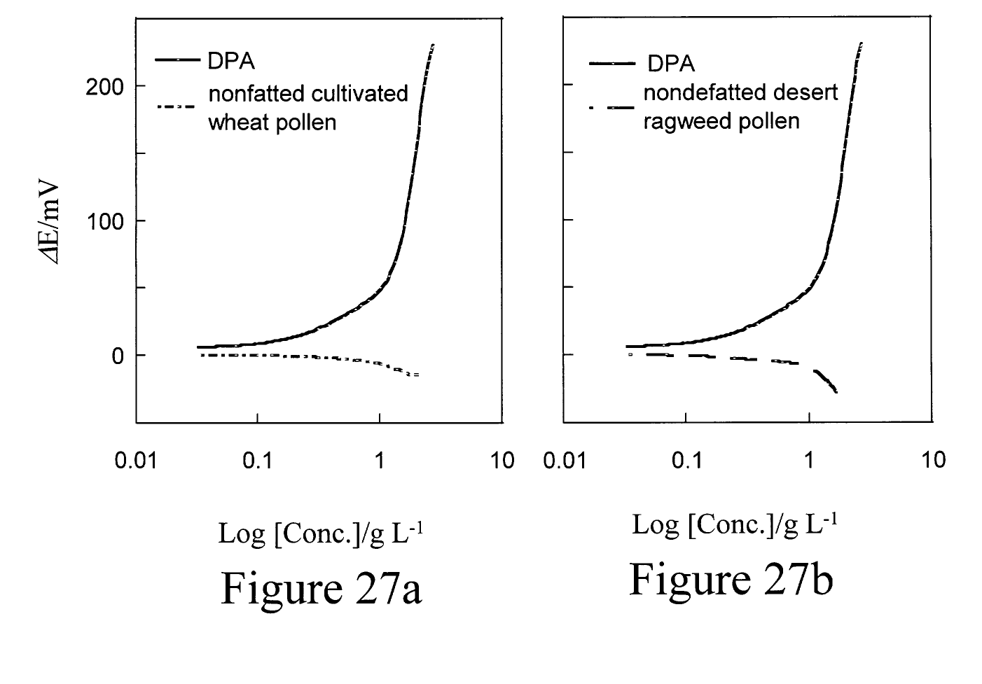

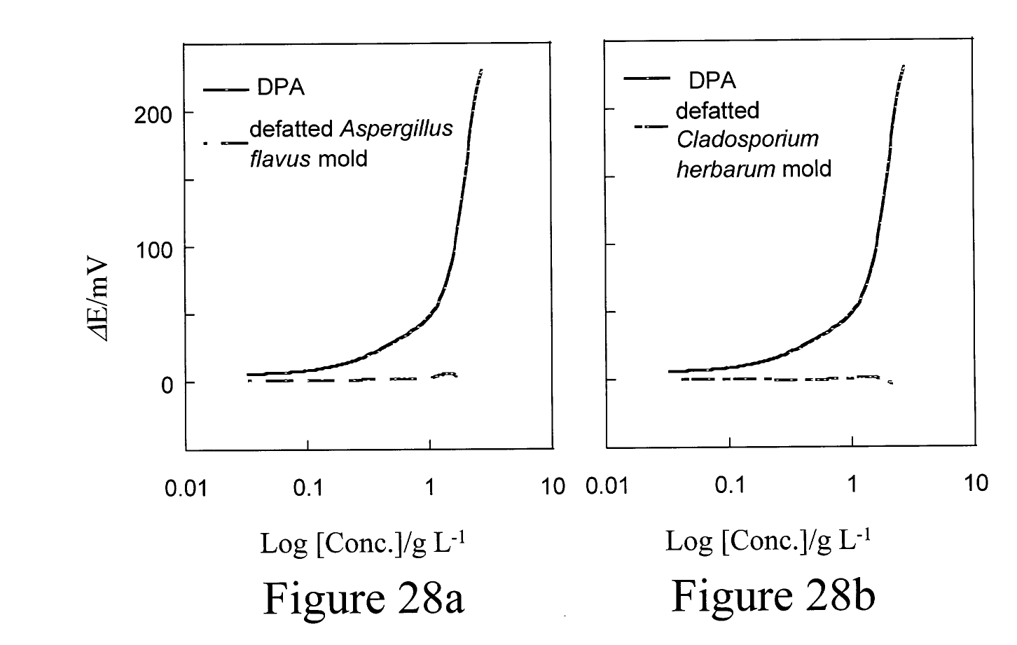

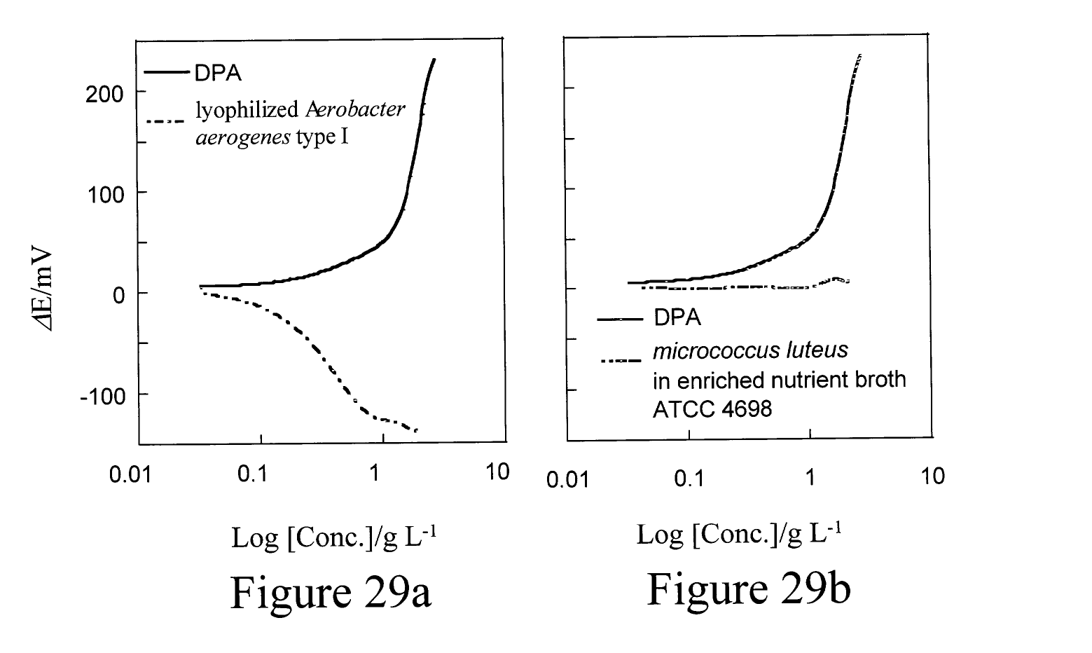

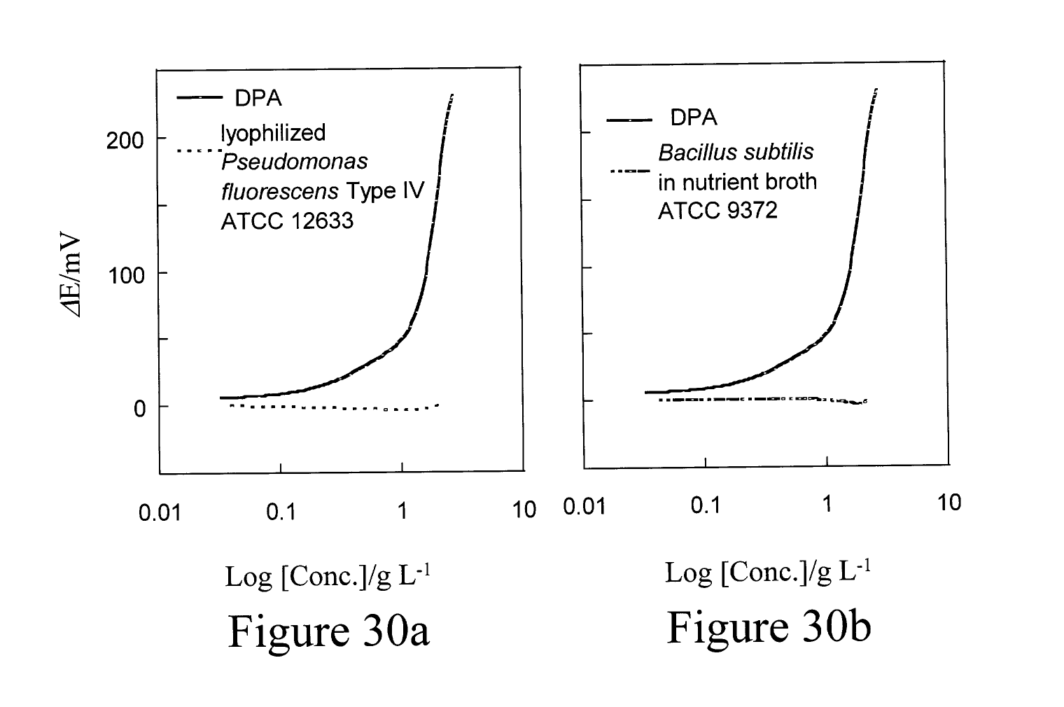

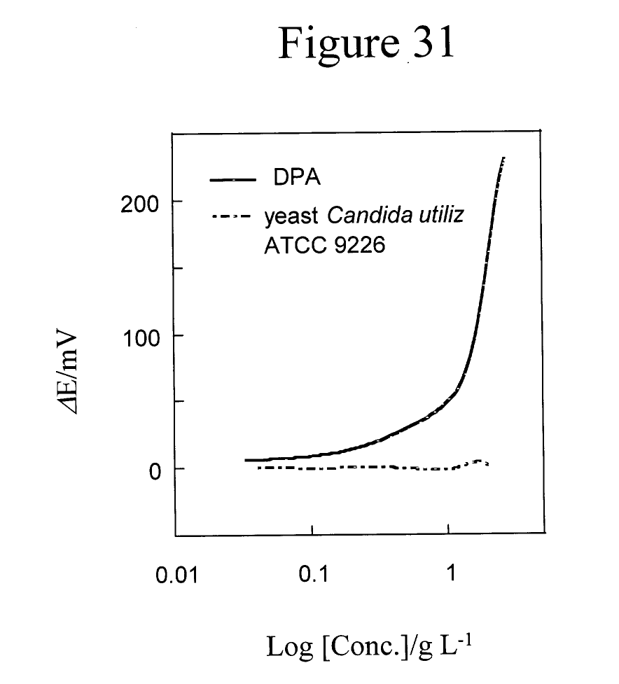

The surface-molecularly imprinted sensor for DPA displayed very specific molecular recognition ability and gave high responses towards DPA. The sensor selectively recognized DPA in the presence of molecules of similar structure, such as benzoic acid. (See FIG. 16.) The surface-molecularly imprinted sensor for DPA also selectively recognized DPA over: inorganic compounds such as NaCl, KCl, K₂HOP₄, NaNO₃, NH₄NO₃, (NH₄)₂SO₄, and CaCO₃ (See FIGS. 17-20a.); organic compounds such as nutrient broth and tryptone, D-Phe, L-glucose, D(+)-malic acid, sodium benzoate, L-tyrosine, DL-tryptophan, riboflavin, and β-NAD (See FIGS. 20b-24.); and biological compounds such as lyophilized B. subtilis, lyophilized Azotobacter vinelandii, defatted German cockroach, nondefatted Eastern cottonwood pollen, nondefatted cultivated wheat pollen, nondefatted desert ragweed pollen, defatted Aspergillus flavus mold, defatted Cladosporium herbarum mold, lyophilized Aeobacter aerogenes Type I, Micrococcus luteus in enriched nutrient broth, lyophilized Pseudomonas fluorescens Type IV, B. subtilis in nutrient broth, and Yeast candida utiliz (See FIGS. 25-31.). The potentiometric selectivity coefficients,

$${K}_{DPA , j}^{POT}$$

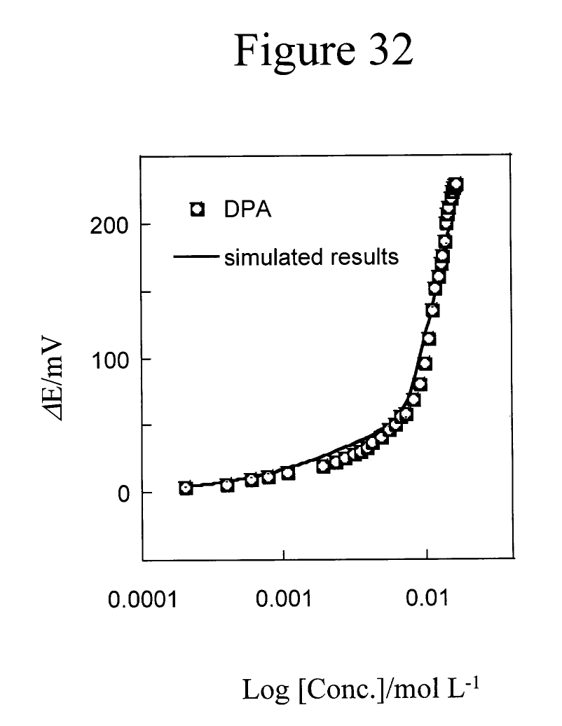

(j: interference), were obtained for DPA sensor based on simulation results (FIG. 32 shows the similarity between simulated and actual results.) and calculated using the following equations:

Nicolsky-Eisenman Equation:

$$E = CONSTANT + \frac{RT}{{z}_{i} F} ln ( {a}_{i} + {\sum }_{j} {K}_{i , j}^{POT} {a}^{{z}_{i} {lz}_{j}} )$$

where E is potential difference between an ion i of charge zᵢ and ion j of charge zⱼ.

$${K}_{DPA , j}^{POT}$$

is the potentiometric selectivity coefficient.

For DPA Sensor:

The selectivity coefficients for inorganic compounds are

$${E}_{DPA} = {E}_{DPA}^{0} + s {log [ {H}^{+} ]}_{0} = {E}_{DPA}^{0} + s log ( [ {DPA}^{+} ] + {K}_{DPA , j}^{POT} [ {a}_{j}^{{z}_{j}} ] )$$

shown in Table 4, below.

TABLE 4
Selectivity Coefficients for Inorganic Compounds

| Compounds | NaCl | KCl | K₂HPO₄ | NaNO₃ | NH₄NO₃ | (NH₄)₂SO₄ | CaCO₃ |
|---|---|---|---|---|---|---|---|
| ${K}_{DPA , j}^{POT}$ | 20 | 44.4 | 4.44 | 0 | 0 | 0 | 2.22 |

The selectivity coefficients for organic compounds are shown in Table 5, below.

TABLE 5
Selectivity Coefficients for Organic Compounds

| | nutrient | | | | D(+)-malic |
|---|---|---|---|---|---|
| Compounds | broth | tryptone | D-Phe | L-glucose | acid |
| ${K}_{DPA , j}^{POT}$ | 0 | 0 | 0 | 0 | 35.56 |
| | sodium | | DL- | | |
| Compounds | benzoate | L-tyrosine | tryptophan | riboflavin | □-NAD |
| ${K}_{DPA , j}^{POT}$ | 0 | 2.22 × 10⁻³ | 2.22 × 10⁻³ | 7.78 | 0 |

The selectivity coefficients for biological materials are shown in Table 6, below.

TABLE 6
Selectivity Coefficients for Organic Compounds

| | | | | nondefatted | nondefatted |
|---|---|---|---|---|---|
| | | lyophilized | defatted | Eastern | cultivated |
| | lyophilized | Azobacter | German | cottonwood | wheat |
| Compounds | B. subtilis | vinelandii | cockroach | pollen | pollen |
| ${K}_{DPA , j}^{POT}$ | 0 | 0 | 0 | 0 | 0 |
| | nondefatted | defatted | defatted | lyophilized | Micrococcus |
| | desert | Aspergillus | Cladosporium | Aerobacter | luteus in |
| | ragweed | flavus | herbarum | aerogenes | enriched |
| Compounds | pollen | mold | mold | Type I | nutrient broth |
| ${K}_{DPA , j}^{POT}$ | 0 | 6.67 × 10⁻³ | 2.22 × 10⁻⁶ | 0 | 3.33 × 10⁻³ |
| | lyophilized | Bacillus | | | |
| | Pseudomonas | globigli in | Yeast | | |
| | fluorescens | nutrient | Candida | | |
| Compounds | Type I | broth | utiliz | | |
| ${K}_{DPA , j}^{POT}$ | 2.22 × 10⁻³ | 2.22 × 10⁻³ | 2.22 × 10⁻² | | |

Except for D(+)-malic acid, none of the substances listed in Tables 4, 5, and 6 yielded any false positive potentiometric response. D(+)-malic acid's false positive response may be attributed to its small size. That is, since D(+)-malic acid is smaller than DPA, it may be able to enter the geometrical cavity created by imprinting with DPA. Even though, only a small fraction must have been able to enter the DPA-imprinted cavities since the potential response for D(+)-malic acid was less than 50 mV. All of the sample concentrations used in these experiments were much higher than they exist in the natural environment.

The surface-molecularly imprinted sensor for DPA's response time, evaluated as the time needed for a 95% signal change, was about 40 s for 0.0157 M DPA. The sensor's reproducibility had average potential variations of 2.79% as a relative standard deviation were observed for consecutive determinations of 0.0105 M DPA. The sensor demonstrated a long lifetime and stability. After measurements were repeated more than 550 times, the response decreased to only 90% of its initial magnitude, demonstrating the long lifetime of the sensors.

§ 4.3.3 MPA Sensor

The proliferation and use of chemical and biological warfare agents has become relevant in our society. Lethal compounds such as Sarin (isopropyl methylphosphonofluoridate), Soman (pinacolyl methylphosphonofluoridate) and VX (o-ethyl-S-2-diisopropylaminoethyl methylphosphonothioate) are highly toxic nerve agents, lethal at low dosages (See, e.g., R. Trapp, SIPRI Chemical & Biological Warfare Studies 3. The Detoxification and Natural Degradation of Chemical Warfare Agents; Taylor & Francis: Philadelphia, Pa., p104 (1985), J. A. F. Compton, Military Chemical and Biological Agents Chemical and Toxicological Properties; The Telford Press: Caldwell, N.J., p458 (1987) p. 458.) and have been used in the recent past. (See, e.g., A. T. Tu, Natural and Selected Synthetic Toxins; American Chemical Society: Washington D.C., Chapter 20, (2000)). Since the nerve agents are hydrolyzed in the environment, their degradation products, such as methylphosphonic acid (MPA) for Sarin, usually are detected for proof of the use of nerve agents. (See, e.g., A. T. Tu, J. Mass Spectrum Soc. Jpn, 44, 293 (1996), P. Hirsjarvi, J. K. Miettinen, J. Paasivirta, E. Kanolahti, (Editors), Trace Analysis of Chemical Warfare Agents, An Approach to the Environmental Monitoring of Nerve Agents, Ministry of Foreign Affairs of Finland, Helsinki, pp 27, 28, 37-39, 59-64, 72-79, 90-99 (1981).)

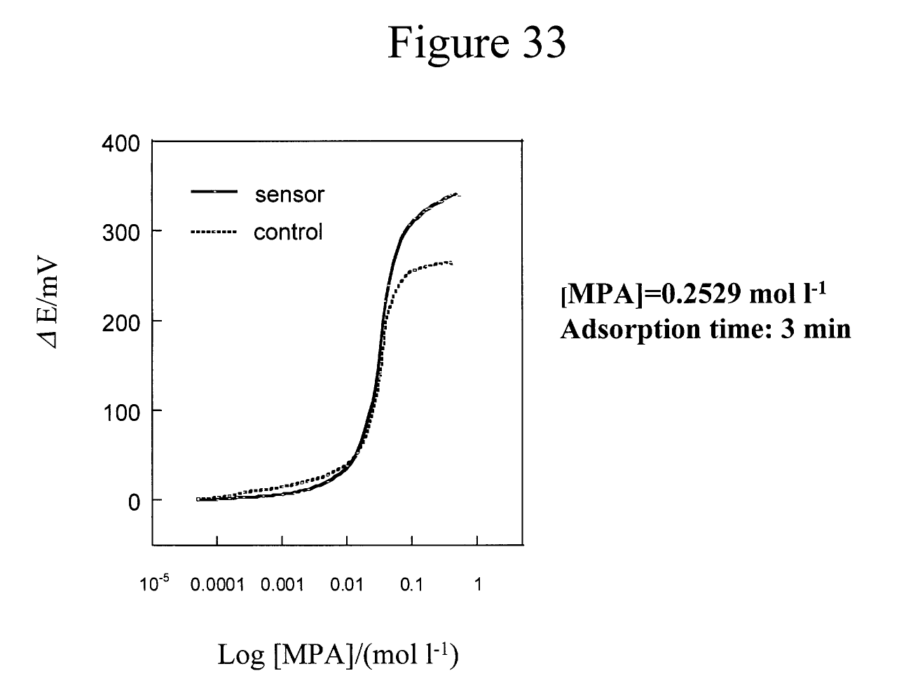

A surface-molecularly imprinted sensor for MPA was fabricated according to the method described in § 4.1.2 above. When 0.2529 M MPA was used during sensor fabrication and adsorption time was 3 minutes, the sensor with surface-molecularly imprinted cavities for MPA recognizes MPA with a potential change as a function of added MPA (FIG. 33, curve (^{●})). MPA was 14.75 times more concentrated on the sensor with surface-molecularly imprinted cavities for MPA than the control sensor. Since MPA was dispersed in a low polarity medium (CHCl₃/CCl₄), the template cavity was initially tailored for undissociated MPA [CH₃(PO) (OH)₂] and programmed not to accept any ionic forms, which were preferentially present in aqueous solution.

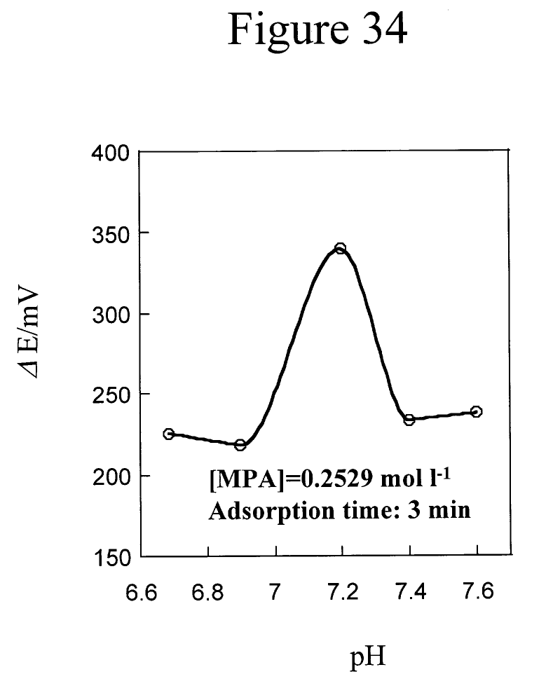

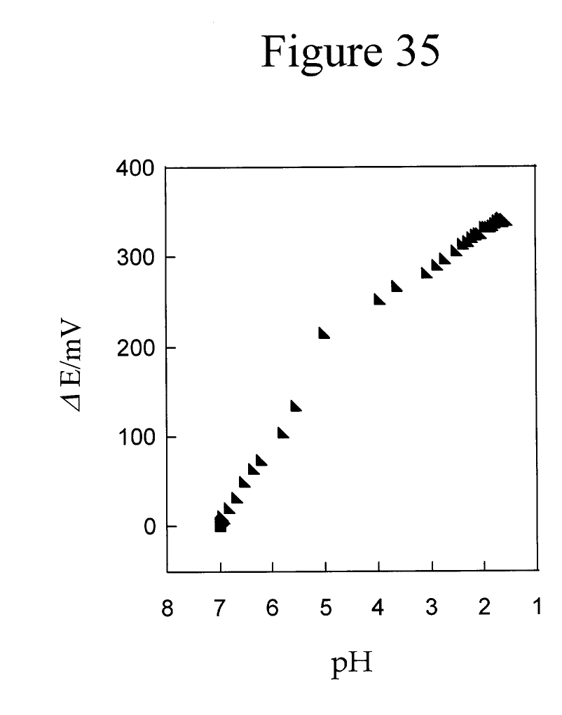

To understand the recognition mechanism more clearly, the pH dependence of the sensor output was analyzed. The pH decreases were coupled with increases in the MPA concentration in phosphate buffer saline, which proves that the sensor responds to protons (See FIGS. 34 and 35). Since the OTS monolayer could only accommodate the neutral MPA, the ITO electrode surface should play an important role in this recognition process because ITO is a nano-scale material (See, e.g., A. K. Kulkarni, K. H. Schulz, T -S. Lim, M. Khan, Thin Solid Film, 308-309, 1-8 (1997).) and has a higher sensitivity and selectivity to hydrogen (See, e.g., U. Weimar, W. Gopel Sensors and Actuators B, 26-27, 13-18 (1995), G. Li, J.; Kawi S. Talanta, 45, 759-766 (1998).).

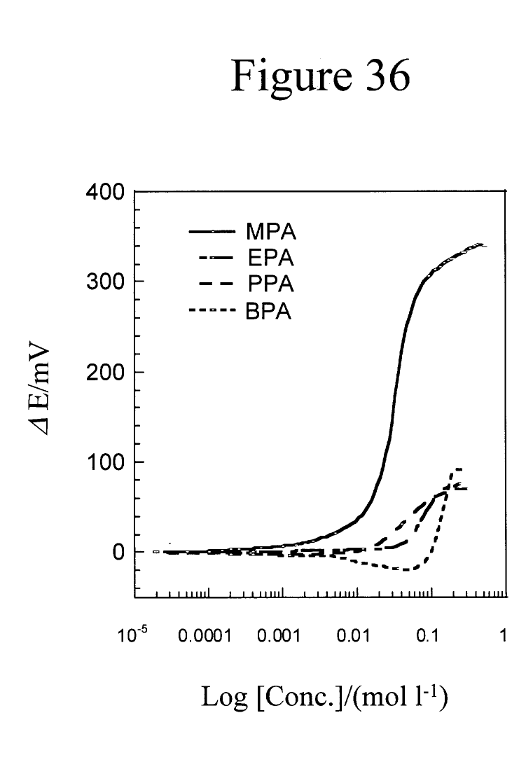

EPA, PPA and BPA are closely related compounds to MPA, and their pKa are also really similar. FIG. 36 shown that all of them gave a less than 100 mV potentiometric response, demonstrating the surface molecularly imprinted MPA sensor had good selectivity characters.

The interaction between the MPA and OTS monolayer/ITO electrode can be divided into two parts: the assembly with OTS with amphiphility in a selected direction; and the electrostatic binding of a proton with the surface oxides. The initial assembly provides the geometrical features for the final recognition site. The electrostatic interaction transfers the chemical interaction energy to the proton-sensitive ITO surface during the molecular recognition in the chemical sensor, as shown in FIG. 3. Thus, the MPA molecules, capable of “carrying” a proton to the ITO surface to change the potential of the ITO electrode, are recognized by the sensor.

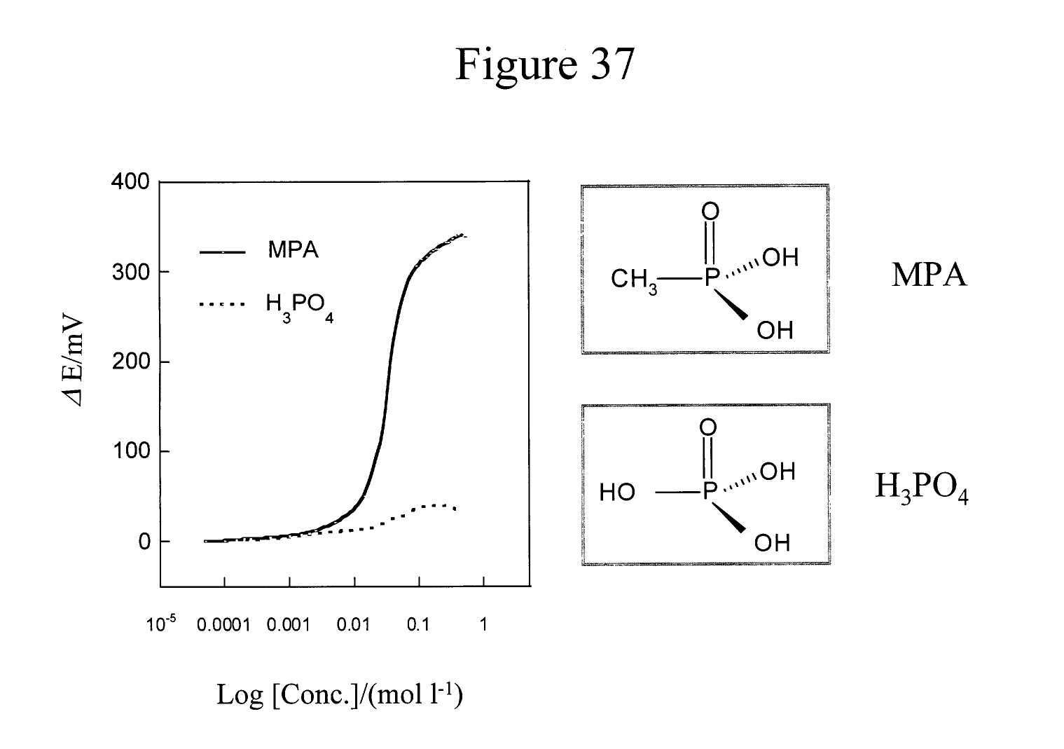

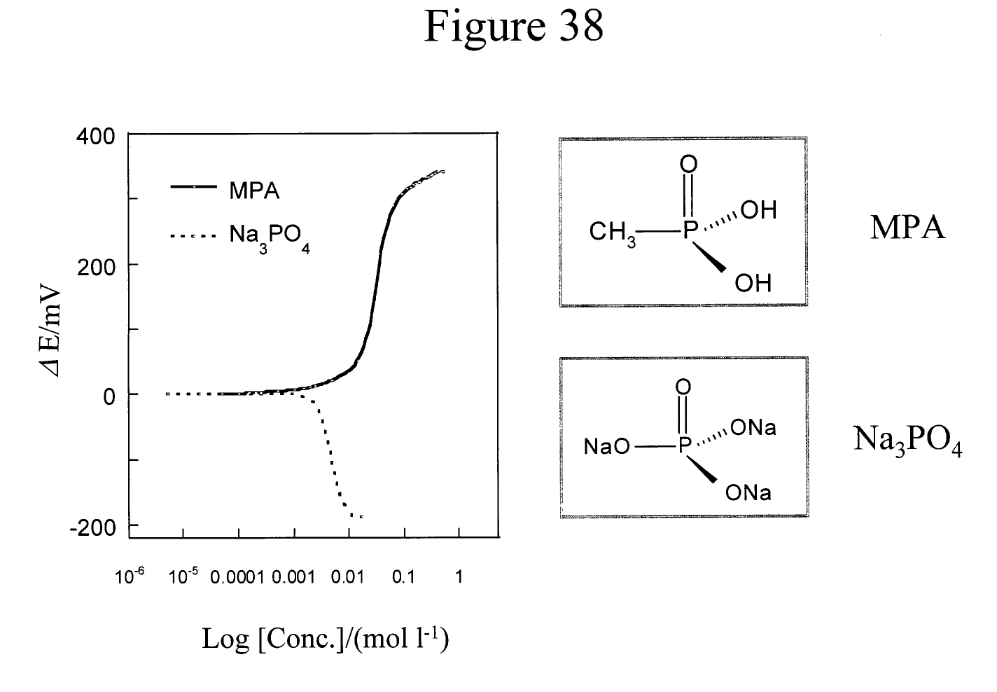

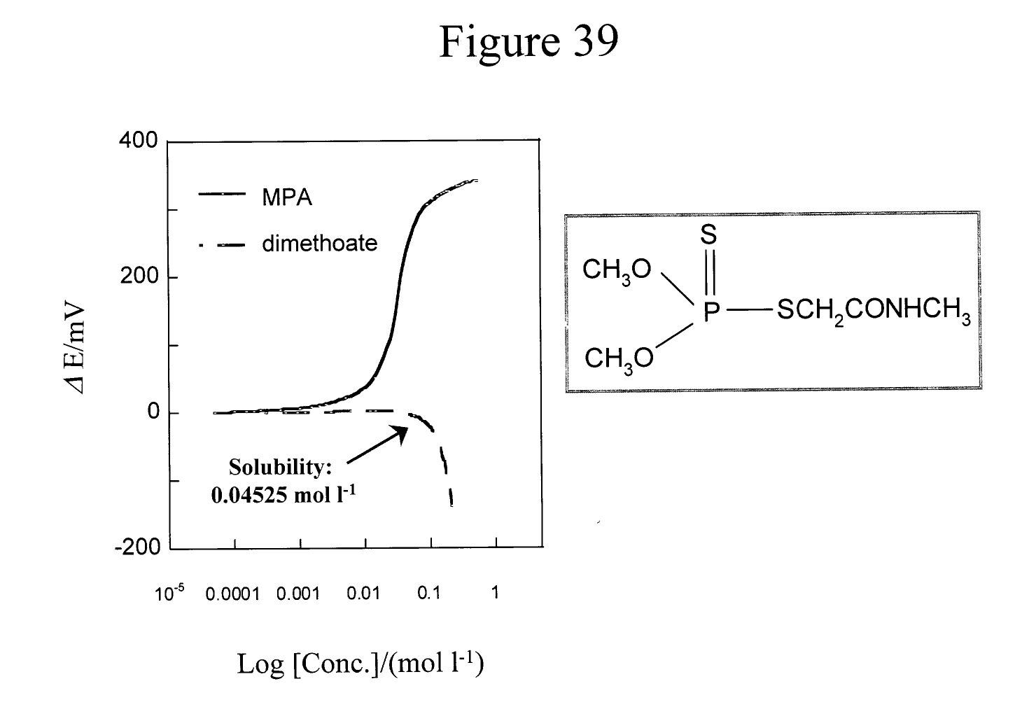

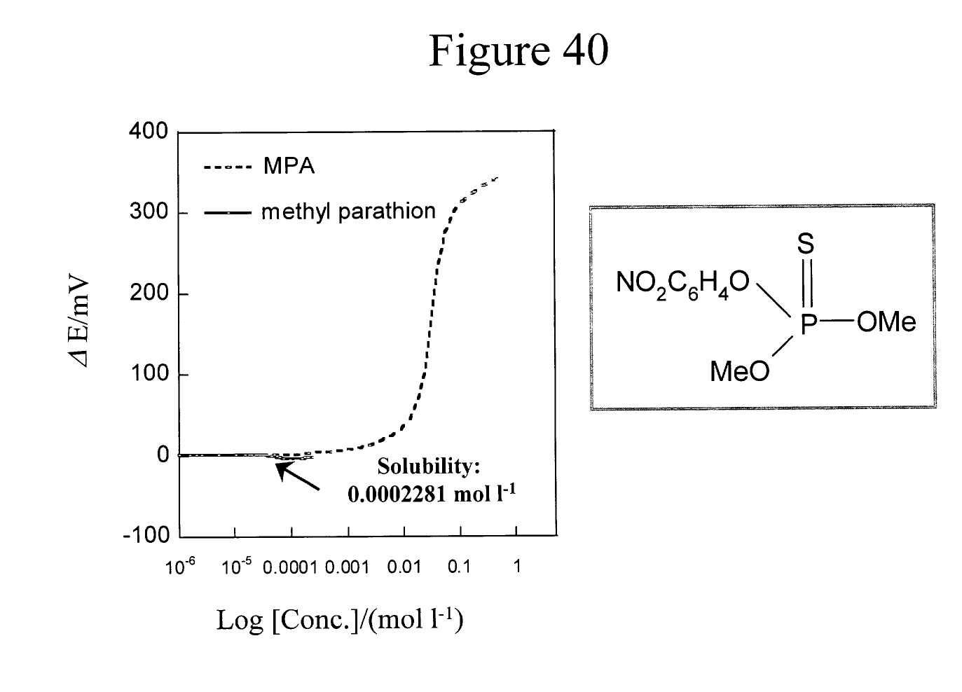

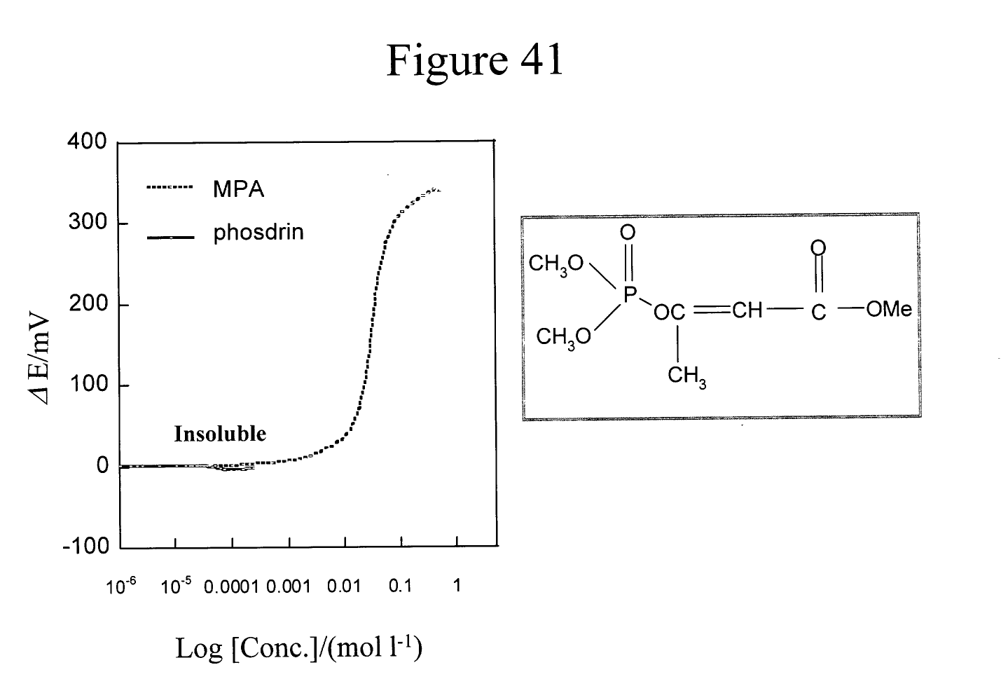

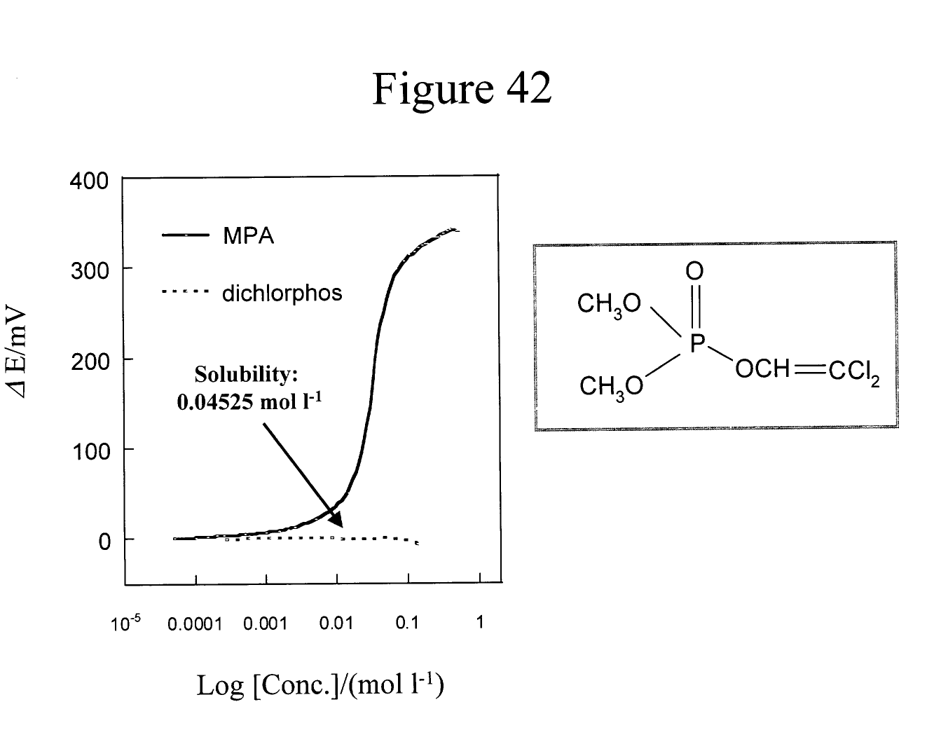

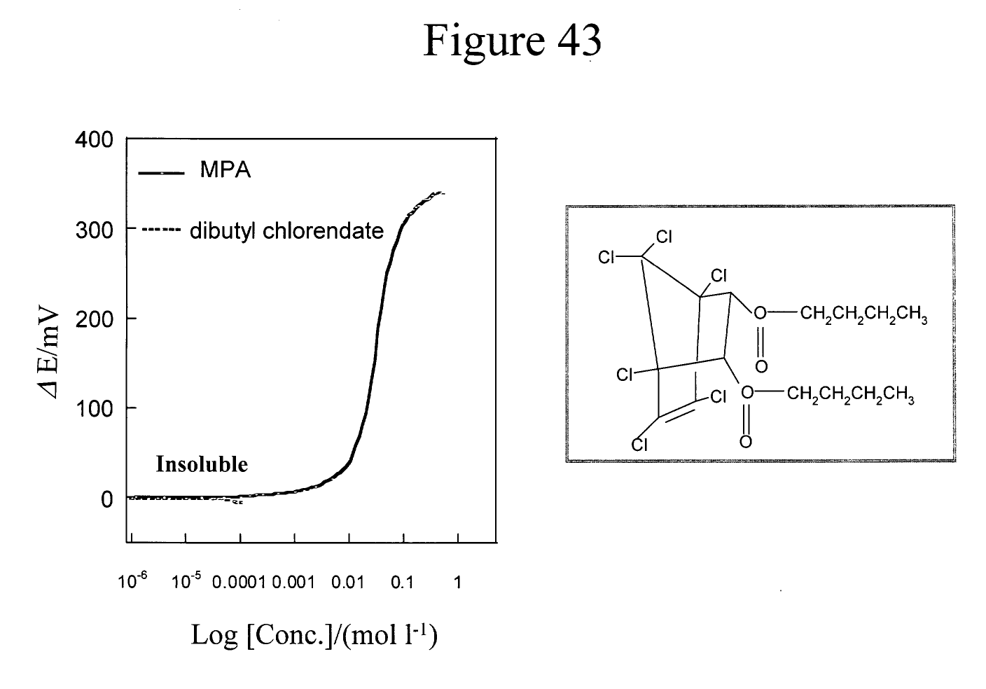

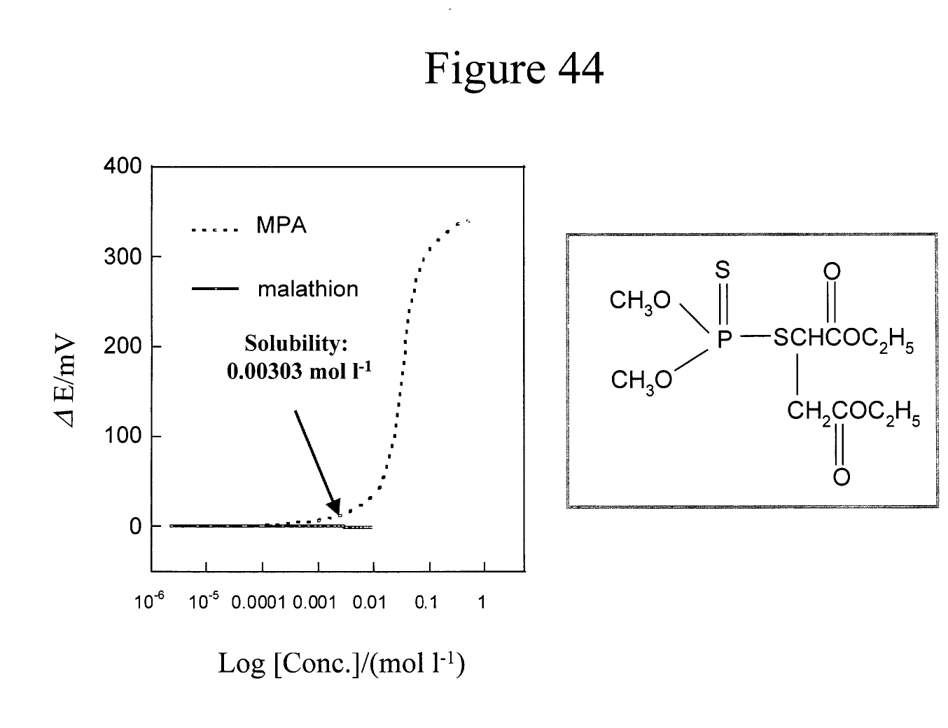

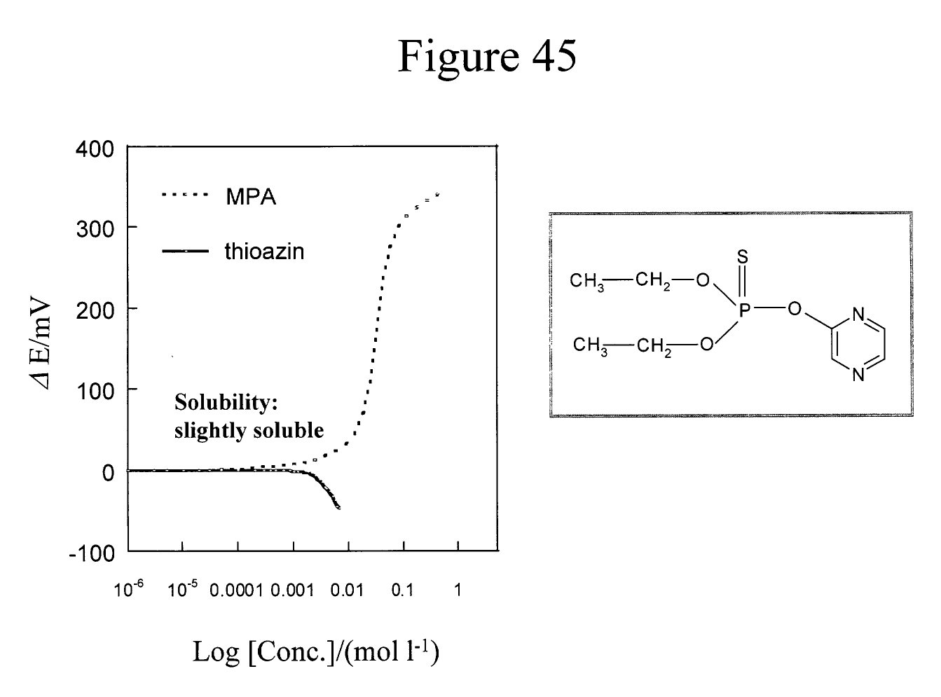

Organophosphorous herbicides and pesticides are chemically analogous to nerve agents and often exist as liquids, oils, or solids at ambient temperatures. Therefore several common pesticides and herbicides, including those similar to the agent Sarin, were tested using the sensor in order to determine the degree of interference from each of them. The surface-molecularly imprinted sensor's sensitivity for MPA was remarkably higher than for other chemically analogous molecules, such as H₃PO₄, Na₃PO₄, dimethoate, methyl parathion, phosdrin, dichlorophos, dibutyl chlorendate, malathion, and thionazin. (See FIGS. 37-46.) These figures also show that the sensor translated the recognition event into a potential change in a concentration range of 4.998×10⁻⁵-0.6154 M with a minimum detectable amount (MDA) of 4.998×10⁻⁵ M.

The potentiometric selectivity coefficient for the MPA-cavity sensor was evaluated by the following equation:

$$E = {E}^{0} + s {log [ {H}^{+} ]}_{0} = {E}^{0} + s log ( [ MPA ] + {K}_{MPA , j}^{POT} [ {a}_{j}^{{Z}_{j}} ] ) \qquad ( 1 )$$

where [H⁺]₀ is the proton concentration on the ITO surface, E and E⁰ are the potentials of the sensor and the standard electrode, respectively, s is the slope, and

$${K}_{MPA , j}^{POT}$$

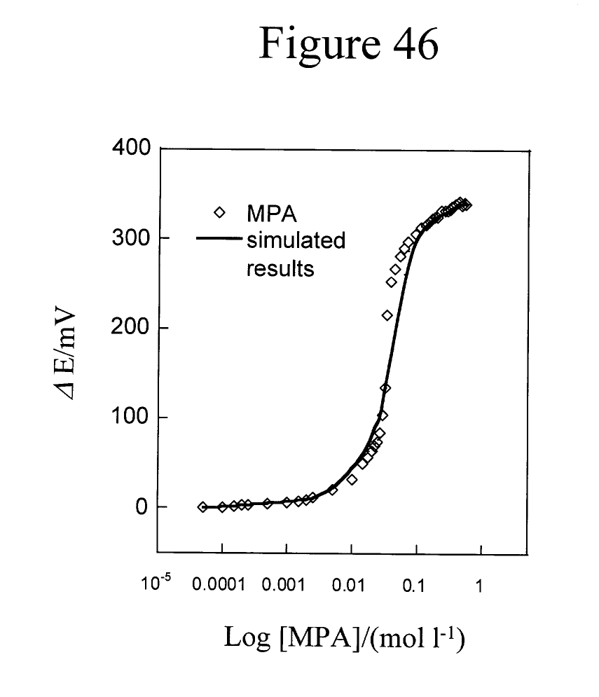

is the potentiometric selectivity coefficient for MPA in the presence of interfering ion, j. (See, e.g., C. M. A. Brett, A. M. O. Brett Electroanalysis; Oxford Science Publications: Oxford University Press, p. 40, (1998).) The output potential for the MPA sensor was simulated by using s values evaluated from the first derivative of the pH response. From this simulation (See FIG. 46.), a

$${K}_{MPA , {H}_{3} {PO}_{4}}^{POT}$$

value of 7.5×10⁻³ has been obtained for the MPA sensor over H₃PO₄, whose structure is very similar to MPA. Based on this value, the sensitivity of the sensor is 133 times as high as that for H₃PO₄, demonstrating remarkable improvement in performance when compared with the previously reported molecularly imprinted polymers. (See, e.g., Molecular and Ionic Recognition with Imprinted Polymers; R. A. Bartsch, M. Maeda, Eds.; American Chemical Society: Washington D.C. (1998), E. Herborg, F. Winquist, I Lundstrom, L. I. Andersson, K. Mosbach, Sensors and Actuators A, 37-38, 296-799 (1993), L. I. Andersson, A. Miyabayashi, D. J. O'Shannessy, K. Mosbach, J. Chromatogr, 516, 323-331 (1990), D. Kirz, O. Ramstrom, A. Svensson, K. Mosbach, K Anal. Chem, 67, 2142-2144 (1995).) Based on the potentiometric selectivity coefficients for other interference molecules (See Table 7, below.), the chemicals that are the most likely to interfere, including H₃PO₄, gave no false positive readings, indicating that the sensor has highly selectivity for MPA.

TABLE 7
Potentiometric selectivity coefficients of MPA
sensor to interferences

| | | | methyl | |
|---|---|---|---|---|
| Interference | H₃PO₄ | Na₃PO₄ | parathion | thionazin |
| ${K}_{DPA , j}^{POT}$ | 7.5 × 10⁻³ | 0 | 0 | 0 |
| dimethoate | phosdrin | dichlorvos | dibutyl | malathion |
| | | | chlorendate | |
| 0 | 3.33 × 10⁻⁴ | 0 | 0 | 0 |

The surface-molecularly imprinted sensor's selectivity comes from its imprinted cavities in the OTS layer. The monolayer acts as a filter that allows molecules of the same size and shape to pass through. After these molecules release protons in electrostatic interaction with the electrode's surface oxides, the sensor yields a potential response. The sensor's response time, evaluated as the time required for a 95% signal response, was about 50 s for 1.5×10⁻² M MPA. This response time is approximately 12 times shorter than luminescent sensors (8 min.) (See A. L. Jenkins, O. M. Uy, and G. M. Murray. Anal. Chem., 1999, 71, 373.) Average potential variations of 2.36% as a relative standard deviation were observed for 11 consecutive determinations of 0.0172 M MPA. After measurements were carried out more than 210 times, the response still remained 92% of its initial magnitude. The sensor's stability is believed to stem from the bio-mimetic molecular imprinting technology used in fabrication.

§ 4.3.4 Other Sensors

The present invention can be used to produce other sensors in which all -trichlorosilane compounds, such as octenyltrichlorosilane, cyclohexlmethyl)trichlorosilane, bromoprpyltrichlorosilane, trichlorosilane, tert-butyltrichlorosilane, ethoxytrichlorosilane, methyltrichlorosilane, pentyltrichlorosilane, etc., which could produce a surface-molecularly imprinted monolayer. Further, other molecular imprinting polymers, such as prepared with protected amino acid benzyloxycarbonyl-L-tyrosine and either 2-vinlpyridine, acrylic or methacrylic acid, or a combination of both, or other self-assembly monolayer, such as alkylthio-compounds, gel, so-gel, hydrogel may be used as the monolayer, polymer film, or three-dimensional matrices. Inorganic, organic, or biological materials may be used as the template. Gold, platinum, glass carbon, graphite, carbon paste, copper, or silver electrodes semi-conductors oxide electrodes such as SnO₂ electrodes, nanocrystalline TiO₂ film electrodes, polymer films such as polyvinyl-alcohol film, silicon wafer, and all the polar solid surfaces may be used as the surface electrode or substrates.

What is claimed is:

1. A surface-molecularly imprinted sensor for detecting target ionic molecules, the sensor comprising:
a) a support surface; and
b) a polymer monolayer or film coating the support surface, wherein the polymer monolayer or film is imprinted with cavities for detecting the target ionic molecules.

2. The surface-molecularly imprinted sensor of claim 1 wherein the support surface is an electrode.

3. The surface-molecularly imprinted sensor of claim 1 wherein the polymer monolayer or film is selected from a group consisting of octadecyltrichlorosilane and any trichlorosilane compounds, the trichlorosilane compounds including octenyltrichlorosilane, cyclohexlmethyl)trichlorosilane, bromoprpyltrichlorosilane, trichlorosilane, tert-butyltrichlorosilane, ethoxytrichlorosilane, methyltrichlorosilane, pentyltrichlorosilane.

4. The surface-molecularly imprinted sensor of claim 1 wherein the cavities are complementary to the size, geometry, and functionality of the target ionic molecules.

5. The surface-molecularly imprinted sensor of claim 1 wherein the cavities are made by template molecules.

6. The surface-molecularly imprinted sensor of claim 5 wherein the template molecules occur in suspension as undisassociated, nonpolar pairs.

7. The surface-molecularly imprinted sensor of claim 1 wherein the cavities imprinted in the polymer monolayer or film are specific to chiral amino acids.

8. The surface-molecularly imprinted sensor of claim 1 wherein the cavities imprinted in the polymer monolayer or film are specific to dipicolinic acid.

9. The surface-molecularly imprinted sensor of claim 1 wherein the cavities imprinted in the polymer monolayer or film are specific to methylphosphonic acid.

10. The surface-molecularly imprinted sensor of claim 1 wherein the cavities imprinted in the polymer monolayer or film are specific to any organic, inorganic, or biological materials used as a template.

11. A method for detecting target ionic molecules using a surface-molecularly imprinted sensor, the method comprising:
a) providing a solution containing target ionic molecules;
b) providing a sensor including a polymer monolayer or film coating a support surface and being imprinted with cavities for detecting the target ionic molecules;
c) choosing a detection method appropriate for the type of support surface; and
d) recognizing target ionic molecules based on the detection method's output.

12. The method of claim 11 wherein the support surface is an electrode.

13. The method of claim 12 wherein the detection method is potentiometry and wherein the detection method's output is the potential response of the solution.

14. The method of claim 12 wherein a hydrophobic interaction occurs between the target molecule and the sensor's imprinted polymer layer and an electrostatic interaction occurs between the target molecule and the sensor's electrode surface.

15. The method of claim 14 wherein the hydrophobic interaction provides selectivity according to the target molecule's size, geometry, and functionality.

16. The method of claim 14 wherein the electrostatic interaction is a proton transfer from the target molecule to the electrode's surface.

17. The method of claim 11 wherein the polymer monolayer is selected from a group consisting of octadecyltrichlorosilane and any trichlorosilane compounds, the tricholorosilane compounds including octenyltrichlorosilane, cyclohexlmethyl)trichlorosilane, bromoprpyltrichlorosilane, trichlorosilane, tert-butyltrichlorosilane, ethoxytrichlorosilane, methyltrichlorosilane, pentyltrichlorosilane.

18. The method of claim 11 wherein the cavities are complementary to the size, geometry, and functionality of the target ionic molecules.

19. The method of claim 18 wherein the cavities are made by template molecules.

20. The method of claim 19 wherein the template molecules occur in suspension as undisassociated, nonpolar pairs.

21. The method of claim 11 wherein the target ionic molecules are chiral molecules.

22. The method of claim 11 wherein the target ionic molecules are chiral amino acids.

22. The method of claim 11 wherein the target ionic molecules are dipicolinic acids.

23. The method of claim 11 wherein the target ionic molecules are methylphosphonic acids.

24. A method for fabricating a surface-molecularly imprinted sensor, the method comprising:
a) co-adsorbing polymer monomers and template molecules on a support surface;
b) polymerizing the polymer monomers while template molecules are adsorbed on the support surface; and
c) removing the template molecules from the sensor's surface.

25. The method of claim 24 wherein the support surface is an electrode.

26. The method of claim 25 wherein the electrode support surface is selected from a group consisting of indium-tin oxide glass electrodes, gold, platinum, glassy carbon, carbon paste, copper, and semiconductor electrodes, and wherein the semiconductor electrodes include SnO₂ and TiO₂.

27. The method of claim 24 wherein the sensor is an optical sensor.

28. The method of claim 27 wherein the sensor's support surface is selected from a group consisting of glass, optic fiber, and quartz.

29. The method of claim 24 wherein the polymer monomers are selected from a group consisting of octadecyltrichlorosilane, octenyltrichlorosilane, cyclohexlmethyl)trichlorosilane, bromoprpyltrichlorosilane, trichlorosilane, tert-butyltrichlorosilane, ethoxytrichlorosilane, methyltrichlorosilane, pentyltrichlorosilane and all alkyl trichlorosilanes.

30. The method of claim 24 wherein the template molecules are chiral molecules.

31. The method of claim 24 wherein the act of co-adsorbing polymer monomers and template molecules on the support surface includes soaking the support in a suspension containing template molecules and polymer monomers.

32. The method of claim 31 wherein the template molecules in suspension occur as undisassociated, nonpolar pairs.

33. The method of claim 24 wherein the act of removing the template molecules from the sensor's surface is performed by solvent extraction.

34. The method of claim 24 wherein the template molecules are chiral amino acids.

35. The method of claim 24 wherein the template molecules are dipicolinic acids.

36. The method of claim 24 wherein the template molecules are methylphosphonic acids.
